(19) United States
(12) Patent Application Publication
(10) Pub. No.: US 2022/0357332 A1
(43) Pub. Date: Nov. 10, 2022

(54) TRIPHENYLENE DERIVATIVES FOR USE IN IMAGING BIOLOGICAL TISSUE OR FLUID

(71) Applicant: CHROMATWIST LIMITED, Birmingham, West Midlands (GB)

(72) Inventors: Jon PREECE, Birmingham, West Midlands (GB); Alex ROBINSON, Birmingham, West Midlands (GB); Owen JONES, Birmingham, West Midlands (GB); Michael BUTLIN, Birmingham, West Midlands (GB); Parvez IQBAL, Birmingham, West Midlands (GB); Sareena SUND, Birmingham, West Midlands (GB)

(21) Appl. No.: 17/763,013

(22) PCT Filed: Sep. 24, 2020
(86) PCT No.: PCT/GB2020/052324

§ 371 Date: Mar. 23, 2022

(30) Foreign Application Priority Data
Sep. 24, 2019 (GB) 1913756.1

(51) Int. Cl. G01N 33/58; G01N 21/64

(52) U.S. Cl. CPC G01N 33/582; G01N 21/6458; C07D 263/54

(57) ABSTRACT

A composition for imaging a biological tissue or fluid comprising a compound of formula (A) and a biologically acceptable diluent or carrier, (A) wherein X represents one of a nitrogen atom, an oxygen atom, a sulphur atom, a phosphorus atom, or a selenium atom; R represents an aromatic group and/or an aliphatic group; p is an integer of 1 to 2; q and s are independently integers of 1, 2, 3, or 4; Y¹, Y², and Y³ independently represent a hydrogen atom, a deuterium atom, a fluorine atom, a chlorine atom, a bromine atom, a substituted or unsubstituted alkyl group, a substituted or unsubstituted aryl group, a polyglycol group, an oxygen atom, a nitrogen atom, a cyano group, a nitro group; and/or wherein two or more of Y¹, Y², and Y³ may combine together to form a condensed ring.

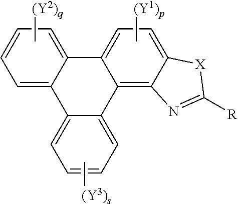

The present invention relates to compositions comprising luminescent compounds, and methods of using said compounds. In particular, the invention relates to the use of luminescent compounds for imaging biological tissue and fluids.

Optical imaging is used to visualise internal tissues and organs within biological organisms, including animals and humans. The aim of tissue imaging may be for laboratory research, to learn about internal structures without surgery or other intrusive techniques. In addition, optical imaging can be used in medicine to detect diseases and other anomalies in internal tissues and organs.

One type of tissue imaging is fluorescence imaging, in which fluorescent dyes, tags or probes are used to mark cellular or molecular structures. The fluorescent dyes, tags or probes can then be imaged by exciting them with illumination light, causing them to fluoresce at a wavelength which is different to the wavelength of the illumination light. The light emitted can then be captured by a microscope or camera.

In some techniques, fluorescent probes are used which bind to a specific target in the organism. For example, immunofluorescence involves the use of a fluorescent molecule chemically conjugated to an antibody which is specific for a target antigen. In other techniques, an organism is genetically modified so that it expresses proteins which are tagged with a fluorescent marker, the most common example being green fluorescent protein (GFP). However, there is a need to label structures in live tissues without affecting cell biology and without modifying the genome of the organism. There is also a need for combinations of fluorochromes that do not cross-talk with each other, and that can be excited by only a few lasers.

The present invention has been devised with these issues in mind.

According to a first aspect of the present invention there is provided a composition for imaging a biological tissue or fluid comprising a luminescent compound of general formula (A) and a biologically acceptable diluent or carrier,

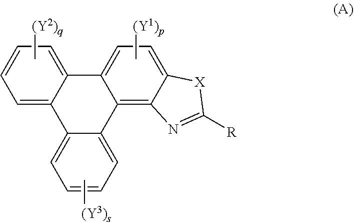

wherein X represents one of a nitrogen atom, an oxygen atom, a sulphur atom, a phosphorus atom, or a selenium atom;

R represents an aromatic group and/or an aliphatic group;

p is an integer of 1 to 2;

q and s are independently integers of 1, 2, 3, or 4;

Y¹, Y², and Y³ independently represent a hydrogen atom, a deuterium atom, a fluorine atom, a chlorine atom, a bromine atom, a substituted or unsubstituted alkyl group, a substituted or unsubstituted aryl group, a polyglycol group (e.g. a group comprising a polyethylene glycol moiety), an oxygen atom (e.g. a hydroxyl group or an alkylated oxygen atom forming an alkoxy group), a nitrogen atom (e.g. a primary, secondary, or tertiary amine group), a cyano group, a nitro group; and/or

wherein two or more of Y¹, Y², and Y³ may combine together to form a condensed ring (e.g. a condensed aromatic ring).

The present inventors have observed that a compound of general formula (A), when perfused into an ex vivo human liver, was effectively taken up by the hepatocytes. Surprisingly, a significantly greater uptake by T cells compared to hepatocytes was observed. Without being bound by theory, the preferential accumulation of the compound in the T cells is thought to have medial utility, such as enabling sites of inflammation to be identified and/or monitored.

Furthermore, the compound was observed to be retained within the hepatocytes and T cells for several hours, enabling visualisation of the hepatic vein. This has not previously been achieved since existing fluorophores quickly leak out of the cells into the blood. The compositions of the invention therefore advantageously enable the structure of tissues to be elucidated.

Thus, in some embodiments the luminescent compound is capable of crossing a cell membrane. The cell may be an animal cell, such as a human cell.

In some embodiments, the luminescent compound is capable of being retained within cells for at least 1 minute, at least 5 minutes, at least 10 minutes, at least 30 minutes, at least 1 hour, or at least 2 hours.

In some embodiments the luminescent compound preferentially accumulates in lymphocytes, such as T cells. In other words, when the luminescent compound is contacted with a mixture of two or more cell types including T cells, the compound is taken up by the T cells in a greater amount than by the other cell(s).

The compounds, compositions and methods described herein may therefore be used to determine the structure of biological tissues, e.g. for research purposes. The compounds, compositions and methods described herein may also be used for assessing the health of biological tissue. For example, the compositions and methods may be used to assess the viability of an ex vivo organ for transplant. Alternatively, the compounds, compositions and methods may be used to detect abnormalities or disease.

The composition of the invention comprises a luminescent compound of general formula (A) in a biologically acceptable diluent or carrier. Suitable biologically-acceptable diluents and carriers include, but are not limited to, physiologically acceptable buffer solutions, buffered saline, e.g. phosphate-buffered saline, saline, balanced crystalloid fluids, deionized water, blood, cell culture medium (e.g. minimal essential medium, or CO₂-independent medium), plasma, and the like. The biologically-acceptable diluent or carrier may be physiologically- or pharmaceutically-acceptable.

The luminescent compound(s) may be dissolved in the diluent or carrier. Alternatively, the luminescent compounds may be dispersed or suspended in, or mixed with the diluent or carrier.

The composition may further comprise an appropriately selected excipient, filler, binder, welting agent, lubricant, surfactant, dispersant, buffer, preservative, adjuvant, antiseptic, analgesic, stabilizer, and/or an isotonic agent.

The composition may be prepared by mixing the luminescent compound with the biologically acceptable diluent or carrier. Prior to mixing, the luminescent compound may be dissolved in a solvent, such as DMSO, THF or 2-methyl THF.

In some embodiments the composition comprises two of more different luminescent compounds, at least one of which has the general formula (A). The two of more different luminescent compounds may be present in the composition at different concentrations, or at the same concentration.

The (or each) luminescent compound may be present in the composition at a concentration of from 0.1 to 20 μg/ml, from 0.5 to 10 μg/ml or from 1 to 5 μg/ml.

For example, the composition may comprise a first luminescent compound of general formula (A), and a second luminescent compound, distinct from the first luminescent compound. In embodiments, the composition may further comprise a third luminescent compound, distinct from the first and second luminescent compounds.

In embodiments, the second and/or third luminescent compound may be a compound distinct from a luminescent compound represented by the general formula (A). In other words, the second and/or third luminescent compound has a structure which is not in accordance with general formula (A). For example, the second and/or third luminescent compound may be a polycyclic aromatic hydrocarbon compound, for example, selected from one of fluorescein, eosin, rhodamine, or an analogue thereof.

In alternative embodiments, the second luminescent compound is represented by the general formula (A) and is distinct from the structure of the first luminescent compound. In some embodiments, the third luminescent compound is represented by the general formula (A) and is distinct from the structure of the first luminescent compound and the second luminescent compound.

Advantageously, the use of different combinations and/or concentrations of distinct first, second, and/or third luminescent compounds, e.g. represented by the general formula (A), provides a bespoke and/or distinct absorption and/or emission spectrum.

Conveniently, the composition may comprise multiple luminescent compounds which are capable of absorbing light at approximately the same wavelength, but which emit light at different wavelengths. In other words, the λₘₐₓ of the compounds is different. This is advantageous since it enables the use of a single laser to excite the compounds.

In some embodiments, the composition comprises a first luminescent compound of formula (A) which emits light at first wavelength (i.e. having a first λₘₐₓ) and a second luminescent compound of formula (A) which emits light at a second wavelength (i.e. having a second λₘₐₓ) which is higher than the first wavelength, wherein both the first and second compounds are capable of absorbing light at a third wavelength which is lower than both the first and second wavelengths.

In some embodiments, the second wavelength (the second λₘₐₓ) is at least 5%, at least 10% or at least 15% higher than the first wavelength (the first λₘₐₓ). This enables the two emissions of the first and second compounds to be easily distinguished.

In some embodiments, both the first and second compounds are capable of absorbing light having a wavelength of 355 nm. In further embodiments, only one of the first and second compounds is capable of absorbing light having a wavelength of 405 nm.

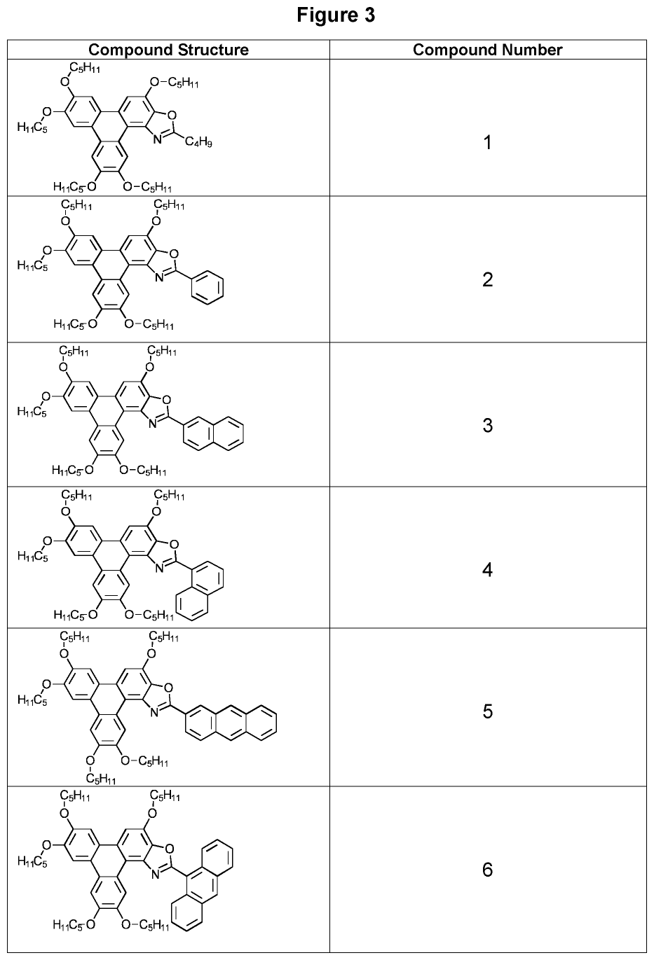

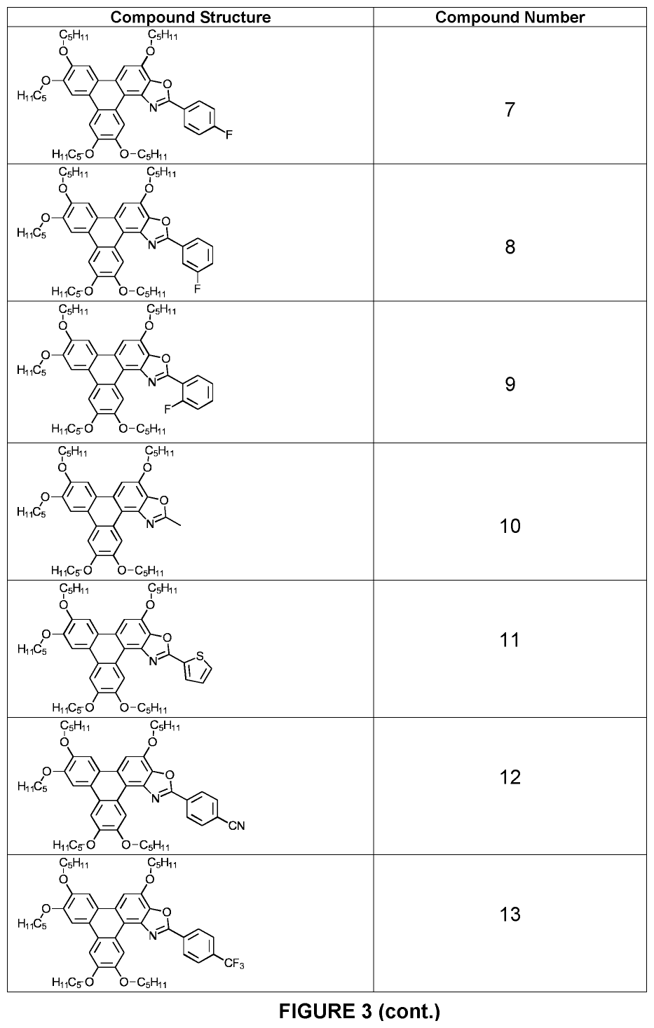

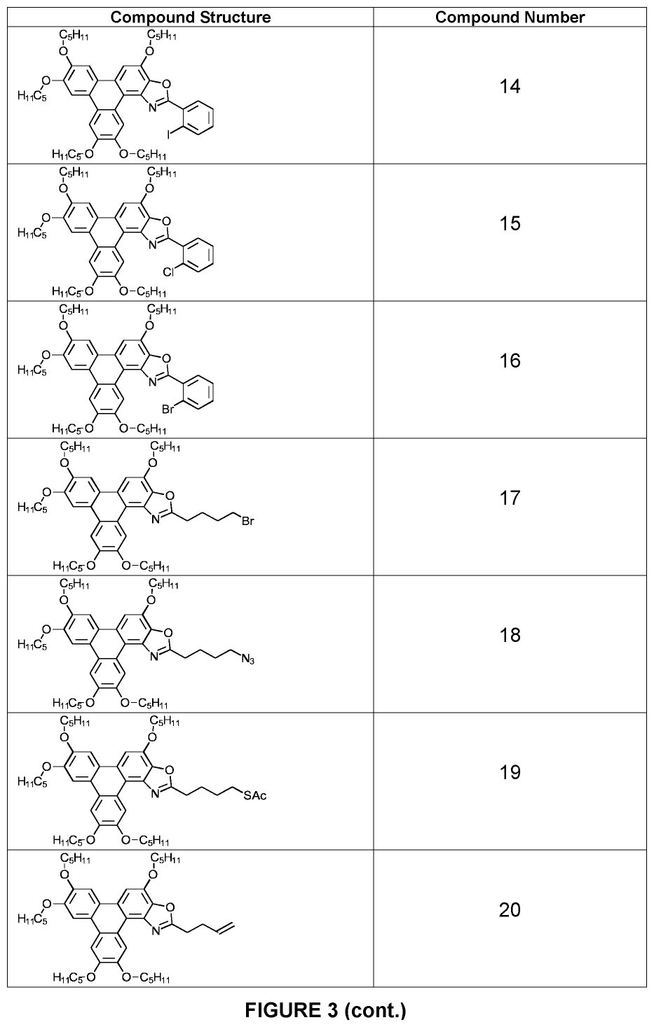

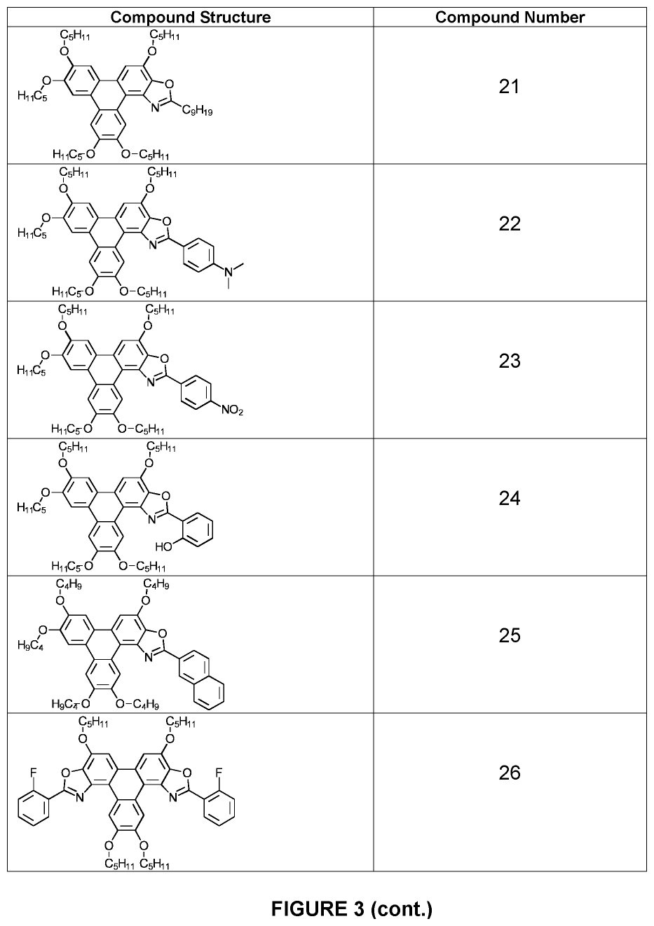

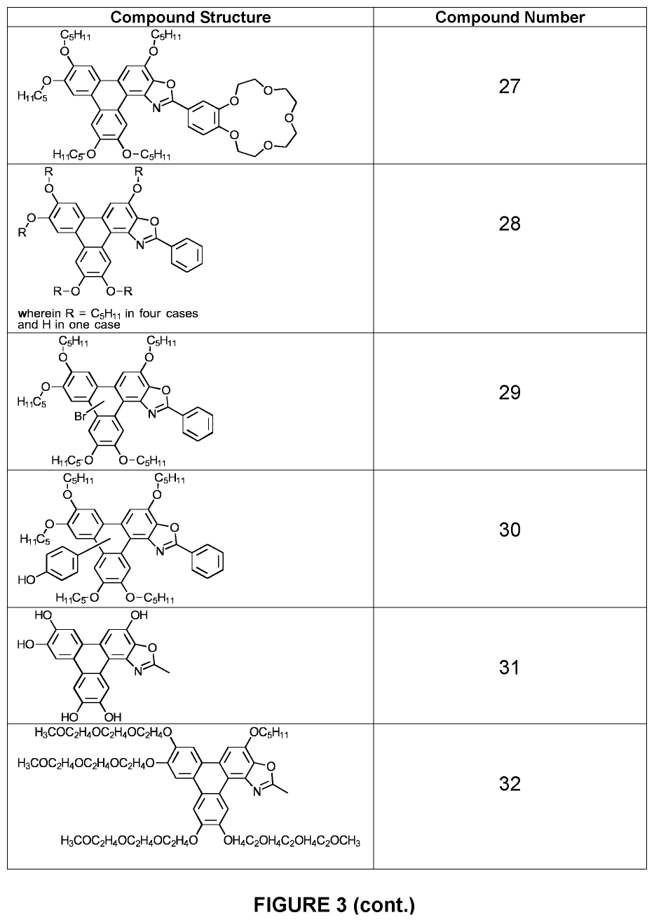

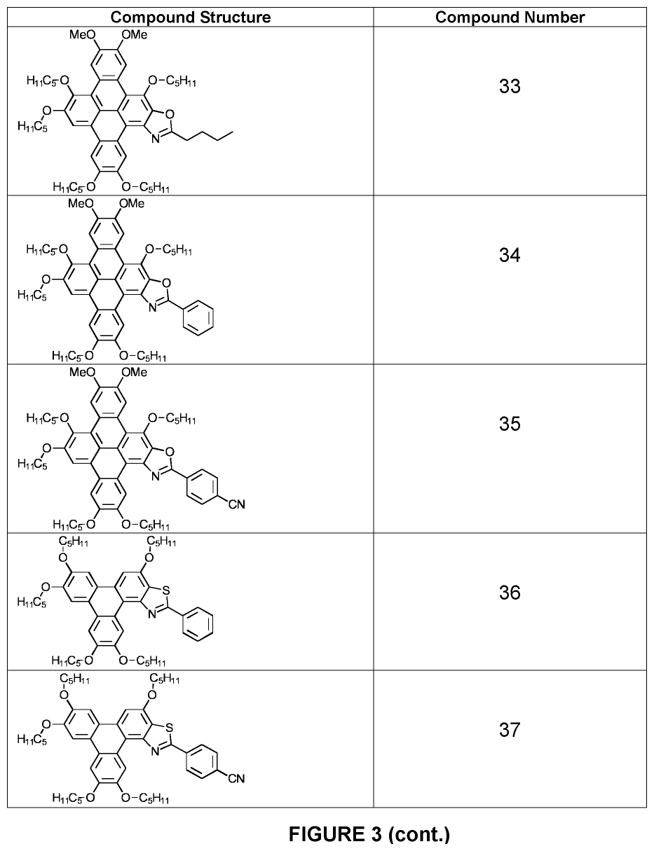

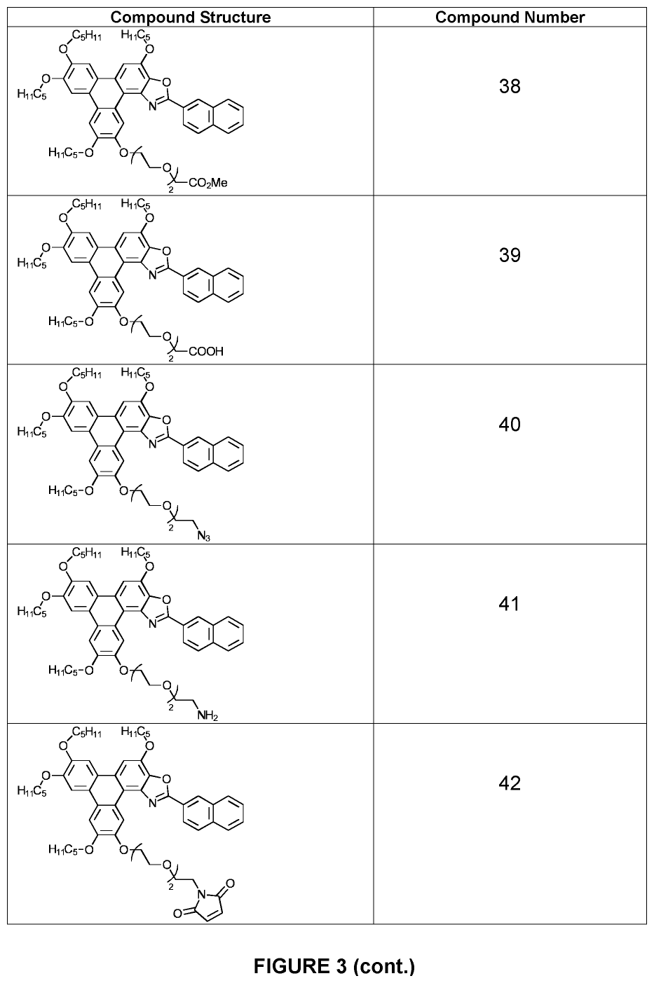

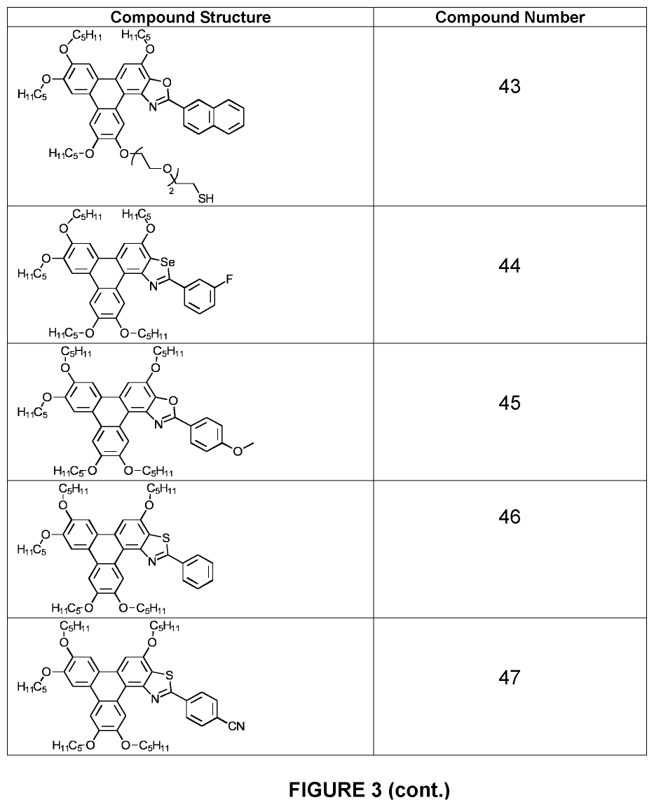

Examples of pairs of luminescent compounds having the above-described properties are: TpOx-Ph-pOMe (Compound No. 45 of FIG. 3) and TpOx-Ph-pCN (Compound No. 12); TpOx-Ph-pOMe (Compound No. 45) and TpOx-2-Nap (Compound No. 3); and TpOx-Ph-pNMe₂ (Compound No. 22) and TpOx-2-Nap (Compound No. 3).

In some embodiments, the luminescent compound of formula (A) is conjugated to a further molecule, such as a protein, a chemical moiety, or a nucleic acid. Such molecules, when conjugated to a luminescent label, may then be used as probes for the detection of a particular target. For example, the composition may comprise a luminescent compound conjugated to an antibody which is specific for a target protein. The composition may then be used for the detection of the target protein in a biological tissue or fluid.

In some embodiments, the composition comprises a first luminescent compound conjugated to a first antibody, and a second luminescent compound which is distinct from the first luminescent compound. The second luminescent compound may be conjugated to a second antibody which is distinct from the first antibody e.g. the first and second antibodies target different proteins.

In some embodiments, the composition comprises cells which have been dyed with the luminescent compound. By “dyed”, it will be appreciated that the luminescent compound may have crossed the cell membrane and reside within the cytoplasm of the cells, or that the luminescent compound may be bound to the cell membrane or a cell-surface receptor, for example via a conjugated antibody or chemical moiety.

The cells may comprise red blood cells and/or white blood cells. The white blood cells may be neutrophils, eosinophils (acidophiles), basophils, lymphocytes (e.g. B cells and/or T cells), and/or monocytes. In some embodiments, the composition comprises T cells which have been dyed with the luminescent compound. Such a composition may be used to monitor the location(s) in a subject in which the T cells accumulate. The T cells may have been obtained from the subject to whom the composition is intended to be administered.

According to a second aspect of the present invention, there is provided the use of a luminescent compound of formula I, or the composition of the first aspect of the invention, in a method of obtaining an image of a biological tissue or fluid.

The image may be obtained by fluorescence imaging, e.g. fluorescence microscopy.

In some embodiments, the biological tissue or fluid is in vivo.

Thus, in a third aspect of the invention, there is provided a method of obtaining an image of a biological tissue or fluid in vivo in a subject, the method comprising:
obtaining an image of the subject using fluorescence microscopy, wherein the subject has been administered a luminescent compound of formula (A), or the composition of the first aspect of the invention.

In some embodiments, the subject has been previously administered the luminescent compound or the composition.

In some alternative embodiments the method further comprises administering to the subject a luminescent compound of formula (A), or the composition of the first aspect of the invention, prior to obtaining the image.

In some embodiments, the method is for analysing healthy biological tissue or fluid in the subject. The luminescent compound or composition may be administered to the body of subject at a location near to a site of interest. For example, in an embodiment wherein the method is for obtaining an image of the liver of the subject, the method may comprise administering the compound or composition to a blood vessel proximal to the liver.

In some alternative embodiments, the biological tissue or fluid is in vitro or ex vivo.

In a fourth aspect of the invention, there is provided a method of obtaining an image of a biological tissue or fluid previously obtained from a subject, the method comprising:
administering to, or contacting with, the biological tissue or fluid a luminescent compound of formula (A), or the composition of the first aspect of the invention; and
obtaining an image of the biological tissue or fluid using fluorescence microscopy.

The compound or composition may be administered immediately prior to obtaining the image. Alternatively, there may be a time delay between the step of administering the compound or composition and the step of obtaining the image. For example, the image may be obtained at least 5 minutes, at least 10 minutes, at least 30 minutes or at least 1 hour after the compound or composition is administered. Such a time delay may be beneficial to enable the luminescent compound(s) to be taken up by cells.

Obtaining an image using fluorescence microscopy may comprise irradiating the subject or tissue or fluid using a light source, and recording an emission spectrum in the UV, visible and/or near infra-red range of the electromagnetic spectrum.

The light source may emit a wavelength that is suitable to excite the luminescent compound(s). The wavelength may be in the range of 100 to 700 nm, from 200 to 600 nm, or from 300 to 500 nm.

The emission spectrum may be recorded in the range of from 100 to 1000 nm, from 100 to 700 nm, from 200 to 600 nm, or from 300 to 500 nm.

The luminescent compound(s) may emit light in the visible or near infra-red spectrum, i.e. between 380 nm and 750 nm and/or may exhibit a Stokes shift of between 8000 cm⁻¹ to 25,000 cm⁻¹, for example, between 15,000 cm⁻¹ to 25,000 cm⁻¹.

In some embodiments, the luminescent compound(s) may exhibit a conductivity value of 5.0×10⁻¹³ S cm⁻¹ and 1.5×10⁻¹¹ S cm⁻¹, for example, between 6×10⁻¹2 S cm⁻¹ and 1.5×10⁻¹¹ S cm⁻¹.

The luminescent compound(s) may exhibit a photoconductivity when irradiated at 350 nm of between 1.5×10⁻¹⁰ S cm⁻¹ and 1×10⁻³ S cm⁻¹, for example, between 1×10⁻⁸ S cm⁻¹ and 1×10⁻³ cm⁻¹.

In some embodiments the fluorescence microscopy is multiphoton microscopy. As is known in the art, in multiphoton microscopy (also known as two-photon microscopy) two photons of light are absorbed for each excitation. This technique differs from traditional fluorescence microscopy in which the excitation wavelength is shorter than the emission wavelength. Two-photon excitation microscopy typically uses near-infrared excitation light. In some embodiments, multiphoton microscopy is carried out by irradiating the subject, tissue or fluid using a light source which emits a wavelength in the range of from 500 to 1000 nm, from 600 to 900 nm or from 700 to 800 nm. The use of multiphoton microscopy is advantageous since it uses lower energy light and is thus less damaging to biological samples. Multiphoton microscopy is also less likely to photobleach the dye, and penetrates further through tissues.

The biological tissue may be any type of tissue, such as connective tissue, muscle tissue (including tendons, ligaments and muscles), nervous tissue, epithelial tissue, vasculature tissue (including arteries, veins and capillaries), lymphoid tissue (including lymphatic vessels and lymph nodes), tissues of the endocrine system, a gland or an organ.

The organ may be a tongue, oesophagus, stomach, small intestine (duodenum, jejunum or ileum), colon, liver, gall bladder, pancreas, heart, lungs, diaphragm, kidney, bladder, ovary, uterus, brain, eye, skin or spleen.

The gland may be the thymus, pituitary gland, pineal gland, parathyroid gland, thyroid gland, salivary gland or adrenal glands.

In some embodiments the fluid is blood.

The compound(s) or composition may be administered topically, orally or parenterally, e.g. by injection or drip infusion. For oral administration, the compound(s) or composition may be formulated as a capsule, a food, a beverage. Capsules may be prepared by filling capsule shells with the compound(s) or composition, either alone or in admixture with one or more accessory ingredients, and then sealing them in the usual manner. A formulation for oral administration may be in the form of a solution, suspension or dispersion in a carrier fluid, such as an aqueous or non-aqueous liquid, or an emulsion.

In some embodiments the compound or composition is administered by injection, for example intravenously, intramuscularly, subcutaneously or intra-arterially.

Formulations suitable for topical administration may be provided for example as gels, solutions, suspensions or dispersions (e.g. in the form of drops or sprays), creams or ointments.

In some embodiments, the compound(s) or composition is administered by perfusion. “Perfusion” refers to the passage of fluid through the circulatory system (e.g. the blood stream) or the lymphatic system to an organ or a tissue. For example, in a method of imaging a biological tissue, such as an ex vivo organ, the compound may be perfused into the organ in a biologically-acceptable carrier fluid, e.g. by injecting the carrier fluid comprising the compound into the blood vessels associated with the organ.

In some embodiments the method comprises administering two or more distinct luminescent compounds to the subject, the biological tissue or fluid. The compounds may be administered simultaneously or sequentially. In some embodiments the compounds are administered by different modes of administration.

In a further aspect, the invention provides a kit for imaging biological tissue or fluid, the kit comprising:
a luminescent compound of formula (A); and
instructions for use.

The kit may further comprise a biologically acceptable diluent or carrier, as described above. Alternatively, or additionally, the kit may comprise one or more further components such as an excipient, a filler, a binder, a wetting agent, a lubricant, a surfactant, a dispersant, a buffer, a preservative, an adjuvant, an antiseptic, an analgesic, and/or an isotonic agent.

The kit may comprise two or more different luminescent compounds, at least one of which has the general formula (A).

In the kit, state of storage of the luminescent compound(s) is not limited. For example, the compound(s) may be stored in liquid (e.g. solution, suspension or dispersion) or solid (e.g. powder, granular or lyophilised) form. The state of storage can be selected by the skilled person according to the stability of the compound(s) and the intended use.

In a further aspect of the invention, there is provided a luminescent compound of formula (A) or the composition of claim 1 for use in a method of diagnosis of a disease or condition in a subject.

The disease or condition may be, but is not limited to: cancer (including cancer of the gastrointestinal tract, reproductive organs (e.g. uterine, ovarian and cervical cancer), liver, kidney, breast, lung, head, mouth, neck, brain and blood (e.g. leukemia, multiple myeloma), inflammation, edema, cardiovascular disorders (such as atherosclerosis), ischemia, autoimmune disease (e.g. rheumatoid arthritis, diabetes, ulcerative colitis, or Crohn's disease), infection (e.g. bacterial, viral, fungal or parasitic infection), skin disease (including skin cancer), eye disease (e.g. macular degeneration, diabetic retinopathy), neurological disorders (e.g. Alzheimer's, stroke) or injury.

The method of diagnosis may comprise administering to the subject a luminescent compound of formula (A), or the composition of the first aspect of the invention; and obtaining an image of the subject using fluorescence microscopy.

Alternatively, the method if diagnosis may comprise administering the luminescent compound of formula (A), or the composition of the first aspect of the invention to a biological tissue or fluid previously obtained from the subject; and obtaining an image of the biological tissue or fluid using fluorescence microscopy.

In some embodiments, the method of diagnosis further comprises comparing the image of the subject, biological tissue or fluid with a reference or image. The reference image may have been obtained from a healthy subject, or it may have been obtained previously from the subject being diagnosed.

In yet a further aspect of the invention, there is provided a method for determining the effectiveness of a treatment or therapy received by a subject.

The subject may be undergoing the treatment or therapy, or they may have completed the treatment or therapy.

The method for determining the effectiveness of a treatment or therapy may comprise obtaining an image of the subject using fluorescence microscopy, wherein the subject has been administered a luminescent compound of formula (A), or the composition of the first aspect of the invention.

In some embodiments, the subject was previously administered the luminescent compound or the composition. In some alternative embodiments, the method further comprises administering to the subject a luminescent compound of formula (A), or the composition of the first aspect of the invention, prior to obtaining the image.

Alternatively, the method may comprise obtaining an image of a biological tissue or fluid obtained from the subject using fluorescence microscopy. The method may further comprise administering to, or contacting with, the biological tissue or fluid a luminescent compound of formula (A), or the composition of the first aspect of the invention.

The method may further comprise comparing the image obtained to a reference image. The reference image may have been obtained from the subject prior to the commencement of treatment or therapy, or earlier during the treatment or therapy.

The invention thus provides a means to monitor the progress of a disease or treatment.

The subject may be an animal. The animal may be a mouse, rat, guinea pig, rabbit, dog, cat, sheep, goat, pig, cow, horse, primate or human. In some embodiments the subject is human.

In embodiments, the luminescent compound(s) (A) may be represented by the following general formula:

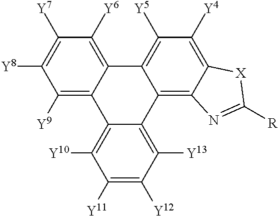

wherein X represents one of a nitrogen atom, an oxygen atom, a sulphur atom, a phosphorus atom, or a selenium atom;
R independently represents an aromatic group and/or an aliphatic group;
Y⁴, Y⁵, Y⁶, Y⁷, Y⁸, Y⁹, Y¹⁰, Y¹¹, Y¹², Y¹³ independently represent a hydrogen atom, a deuterium atom, a fluorine atom, a chlorine atom, a bromine atom, a substituted or unsubstituted alkyl group, a substituted or unsubstituted aryl group, a polyglycol group (e.g. a group comprising a polyethylene glycol moiety), an oxygen atom (e.g. a hydroxyl group or an alkylated oxygen atom forming an alkoxy group), a nitrogen atom (e.g. a primary, secondary, or tertiary amine group), a cyano group, a nitro group; and/or
wherein two or more of Y⁴, Y⁵, Y⁶, Y⁷, Y⁸, Y⁹, Y¹⁰, Y¹¹, Y¹², Y¹³ combine together to form a condensed ring (e.g. a condensed aromatic ring).

In embodiments, the luminescent compound(s) may be a triphenylene derivative. In alternative embodiments, the luminescent compound(s) may comprise a fused polycyclic aromatic hydrocarbon comprising six 6-membered rings.

In embodiments, Y⁸ represents an oxygen atom and Y⁹ represents a nitrogen atom, Y⁸ and Y⁹ being bonded to form an oxazole moiety comprising an R group selected from an aromatic group and/or an aliphatic group.

In embodiments, Y⁵ and Y⁶ represent carbon atoms that combine together to form a condensed ring, e.g. a condensed aromatic ring.

In embodiments, the luminescent compound(s) may be represented by the following general formula:

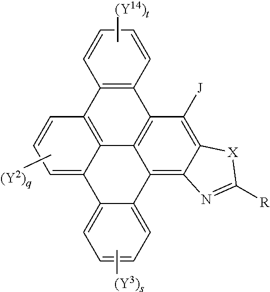

wherein X represents one of a nitrogen atom, an oxygen atom, a sulphur atom, a phosphorus atom, or a selenium atom;
R independently represents an aromatic group and/or an aliphatic group;
q is independently an integer of 1 to 3;
s is independently an integer of 1 to 4;
is independently an integer of 1 to 4;
Y², Y³, and Y¹⁴ and J independently represent a hydrogen atom, a deuterium atom, a fluorine atom, a chlorine atom, a bromine atom, a substituted or unsubstituted alkyl group, a substituted or unsubstituted aryl group, a polyglycol group (e.g. a group comprising a polyethylene glycol moiety) an oxygen atom (e.g. a hydroxyl group or an alkylated oxygen atom forming an alkoxy group), a nitrogen atom (e.g. a primary, secondary, or tertiary amine group), a cyano group, a nitro group; and/or
wherein two or more of Y¹, Y², and Y¹⁴ may combine together to form a condensed ring (e.g. a condensed aromatic ring).

In embodiments, the luminescent compound(s) may be represented by the following general formula:

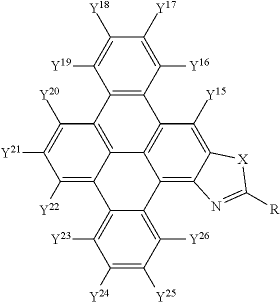

wherein X represents one of a nitrogen atom, an oxygen atom, a sulphur atom, a phosphorus atom, or a selenium atom;
R independently represents an aromatic group and/or an aliphatic group;
Y¹⁵, Y¹⁶, Y¹⁷, Y¹⁸, Y¹⁹, Y²⁰, Y²¹, Y²², Y²³, Y²⁴, Y²⁵, Y²⁶ independently represent a hydrogen atom, a deuterium atom, a fluorine atom, a chlorine atom, a bromine atom, a substituted or unsubstituted alkyl group, a substituted or unsubstituted aryl group, a polyglycol group (e.g. a group comprising a polyethylene glycol moiety) an oxygen atom (e.g. a hydroxyl group or an alkylated oxygen atom forming an alkoxy group), a nitrogen atom (e.g. a primary, secondary, or tertiary amine group), a cyano group, a nitro group; and/or
wherein two or more of Y¹⁵, Y¹⁶, Y¹⁷, Y¹⁸, Y¹⁹, Y²⁰, Y²¹, Y²², Y²³, Y²⁴, Y²⁵, Y²⁶ combine together to form a condensed ring (e.g. a condensed aromatic ring).

In some embodiments, Y²¹ represents an oxygen atom and Y²² represents a nitrogen atom, Y²¹ and Y²² being bonded to form an oxazole moiety comprising an R group selected from an aromatic group and/or an aliphatic group.

In embodiments, the luminescent compound(s) are represented by the following general formula:

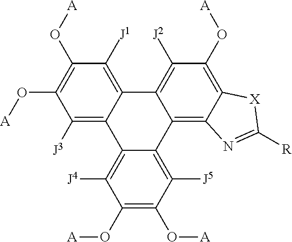

wherein X represents one of a nitrogen atom, an oxygen atom, a sulphur atom, a phosphorus atom, or a selenium atom;
R independently represents an aromatic group and/or an aliphatic group;
A independently represents a hydrogen atom, an aryl group, an alkyl group (e.g. an alkyl ether) comprising 1 to 20 carbons (e.g. 1 to 15 carbons, or 1 to 10 carbons, or 10, 9, 8, 7, 6, 5, 4, 3, 2 or 1 carbons);
J¹, J², J³, J⁴, J⁵ independently represent a hydrogen atom, a deuterium atom, a fluorine atom, a chlorine atom, a bromine atom, a substituted or unsubstituted alkyl group, a substituted or unsubstituted aryl group, a polyglycol group (e.g. a group comprising a polyethylene glycol moiety) an oxygen atom (e.g. a hydroxyl group or an alkylated oxygen atom forming an alkoxy group), a nitrogen atom (e.g. a primary, secondary, or tertiary amino group), a cyano group, a nitro group; and/or
wherein J¹ and J² may combine together to form a condensed ring (e.g. a condensed aromatic ring).

In embodiments, A independently comprises or consists of an alkyl group, for example, a straight chain alkyl group. In embodiments, A is independently selected from one or more of a CH₃, C₂H₅, C₃H₇, C₄H₉, C₅H₁₁, C₆H₁₃, C₇H₁₅, OC₈H₁₇, C₉H₁₉, or C₁₀H₂₁ group. In embodiments, A may independently represent C₅H₁₁ and/or C₄H₉. In embodiments, A may independently represent C₅H₁₁ and/or C₄H₉.

In embodiments, A may independently represent a polyethylene glycol (PEG) group (e.g. C₂H₄OC₂H₄OC₂H₄OCH₃).

In embodiments, A may independently represent an alkyl group comprising a reactive functional group FG, for example, A may independently represent an alkyl group or a polyether group comprising a carboxylic acid moiety, an ester, an azide, an amine, a maleimide, and/or a thiol moiety. In embodiments, A independently represents (CH₂CH₂O)₂CH₂CH₂FG moiety, wherein FG is selected from one of a carboxylic acid moiety, an ester, an azide, an amine, a maleimide, and/or a thiol moiety.

In embodiments, X represents an oxygen atom. In embodiments, X represents a sulphur atom.

In embodiments, J¹, J², J³, J⁴, J⁵ independently represent a hydrogen atom or a deuterium atom.

In embodiments, R represents an aliphatic group or moiety. In embodiments, R represents an aromatic group or moiety.

In embodiments, the triphenylene derivative may not be the compound wherein A is C₅H₁₁, J is H and R is C₄H₉.

In embodiments, the luminescent compound(s) are represented by the following general formula:

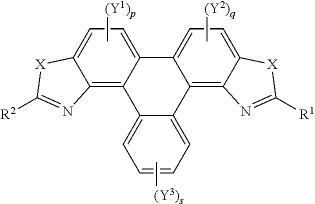

wherein X represents one of a nitrogen atom, an oxygen atom, a sulphur atom, a phosphorus atom, or a selenium atom;
R¹ and R² independently represents an aromatic group and/or an aliphatic group;
p and q are independently an integer of 1 to 2;
s is an integer of 1 to 4;
Y¹, Y², and Y³ independently represent a hydrogen atom, a deuterium atom, a fluorine atom, a chlorine atom, a bromine atom, a substituted or unsubstituted alkyl group, a substituted or unsubstituted aryl group, a polyglycol group (e.g. a group comprising a polyethylene glycol moiety) an oxygen atom (e.g. a hydroxyl group or an alkylated oxygen atom forming an alkoxy group), a nitrogen atom (e.g. a primary, secondary, or tertiary amine group), a cyano group, a nitro group; and/or
wherein two or more of Y¹, Y², and Y³ may combine together to form a condensed ring (e.g. a condensed aromatic ring).

In embodiments, the luminescent compound(s) are represented by the following general formula:

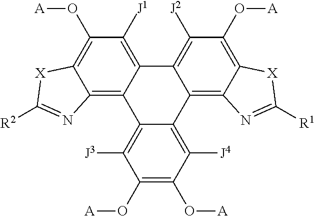

wherein X represents one of a nitrogen atom, an oxygen atom, a sulphur atom, a phosphorus atom, or a selenium atom;
R¹ and R² independently represents an aromatic group or an aliphatic group;
A independently represents a hydrogen atom, an aryl group, an alkyl group (e.g. an alkyl ether) comprising 1 to 20 carbons (e.g. 1 to 15 carbons, or 1 to 10 carbons, or 10, 9, 8, 7, 6, 5, 4, 3, 2 or 1 carbons);
J¹, J², J³, J⁴ independently represent a hydrogen atom, a deuterium atom, a fluorine atom, a chlorine atom, a bromine atom, a substituted or unsubstituted alkyl group, a substituted or unsubstituted aryl group, a polyglycol group (e.g. a group comprising a polyethylene glycol moiety) an oxygen atom (e.g. a hydroxyl group or an alkylated oxygen atom forming an alkoxy group), a nitrogen atom (e.g. a primary, secondary, or tertiary amine group), a cyano group, a nitro group; and/or
wherein J¹ and J² may combine together to form a condensed ring (e.g. a condensed aromatic ring).

In all embodiments, the term “condensed ring” is intended to define groups that bond together to form a ring (e.g. an aromatic ring, and/or a heterocyclic ring) that forms part of the core of the luminescent compound, that is, to expand the triphenylene core.

In embodiments, A may independently represent C₅H₁₁ and/or C₄H₉. In embodiments, A may independently represent a polyethylene glycol (PEG) group (e.g. C₂H₄OC₂H₄OC₂H₄OCH₃). In embodiments, A may independently represent an alkyl group comprising a reactive functional group FG, for example, A may independently represent an alkyl group comprising a carboxylic acid moiety, an ester, an azide, an amine, a maleimide, and/or a thiol moiety. In embodiments, A independently represents —(CH₂CH₂O)₂CH₂CH₂FG or (CH₂CH₂O)₂CH₂FG moiety, wherein FG is selected from one of a carboxylic acid moiety, an ester, an azide, an amine, a maleimide, and/or a thiol moiety.

In embodiments, the luminescent compound(s) are represented by the following general formula:

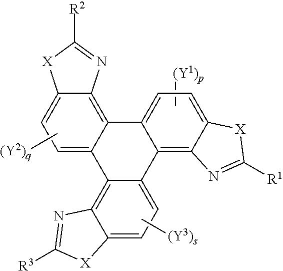

wherein X represents one of a nitrogen atom, an oxygen atom, a sulphur atom, a phosphorus atom, or a selenium atom;
R¹, R², R³ independently represent an aromatic group or an aliphatic group;
p, q, and s are each independently an integer of 1 to 2;
Y¹, Y², and Y³ independently represent a hydrogen atom, a deuterium atom, a fluorine atom, a chlorine atom, a bromine atom, a substituted or unsubstituted alkyl group, a substituted or unsubstituted aryl group, a polyglycol group (e.g. a group comprising a polyethylene glycol moiety) an oxygen atom (e.g. a hydroxyl group or an alkylated oxygen atom forming an alkoxy group), a nitrogen atom (e.g. a primary, secondary, or tertiary amine group), a cyano group, a nitro group; and/or
wherein two or more of Y¹, Y², and Y³ may combine together to form a condensed ring (e.g. a condensed aromatic ring).

In embodiments, the luminescent compound(s) are represented by the following general formula:

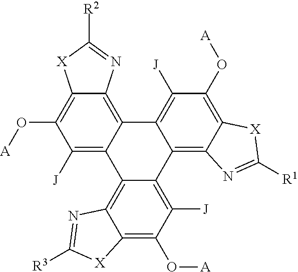

wherein X represents one of a nitrogen atom, an oxygen atom, a sulphur atom, a phosphorus atom, or a selenium atom;
R¹, R², R³ independently represent an aromatic group and/or an aliphatic group;
A independently represents a hydrogen atom, an aryl group, an alkyl group (e.g. an alkyl ether) comprising 1 to 20 carbons (e.g. 1 to 15 carbons, or 1 to 10 carbons, or 10, 9, 8, 7, 6, 5, 4, 3, 2 or 1 carbons);
J independently represent a hydrogen atom, a deuterium atom, a fluorine atom, a chlorine atom, a bromine atom, a substituted or unsubstituted alkyl group, a substituted or unsubstituted aryl group, a polyglycol group (e.g. a group comprising a polyethylene glycol moiety) an oxygen atom (e.g. a hydroxyl group or an alkylated oxygen atom forming an alkoxy group), a nitrogen atom (e.g. a primary, secondary, or tertiary amino group), a cyano group, a nitro group; and/or
wherein J may combine together to form a condensed ring (e.g. a condensed aromatic ring).

In embodiments, X represents an oxygen atom. In embodiments, X represents a sulphur atom.

In embodiments, A independently comprises or consists of an alkyl group, for example, a straight chain alkyl group. In embodiments, A is independently selected from one or more of a CH₃, C₂H₅, C₃H₇, C₄H₉, C₅H₁₁, C₆H₁₃, C₇H₁₅, OC₈H₁₇, C₉H₁₉, or C₁₀H₂₁ group.

In embodiments, J, J¹, J², J³, J⁴, J⁵ independently represent a hydrogen atom.

In embodiments, J, J¹, J², J³, J⁴, J⁵ independently represent a deuterium atom.

In embodiments, J, J¹, J², J³, J⁴, J⁵ independently represent a heteroatom (e.g. a nitrogen atom, an oxygen atom, a halogen, e.g. F, Cl, Br, I).

In embodiments, A comprises further functionality, for example, A may further comprise fluorine atoms, chlorine atoms, cyano groups, nitro groups, glycol, alkoxy, thioalkoxy, polyethylene glycol, amino, acetate, carboxylic acid, amide, thioamide, thioester, azo, and/or silyl groups. In embodiments, A comprises a functional group capable of forming a covalent bond with a second molecule, e.g. a biomolecule or a small molecule, for example, a drug molecule. The functional group may be selected from, for example, a carboxylic acid, an ester, an azide, an amine, a maleimide, a thiol, an isothiocyanate, an aldehyde, and/or an aliphatic alcohol. In embodiments, the functional group may be located at the terminus of one or more of A.

In embodiments, J comprises or represents an aryl group, e.g. a phenol group. Additionally or alternatively, J comprises a halogen atom, e.g. fluorine, chlorine, bromine, or iodine.

In embodiments, R, R¹, R², and/or R³ may be an alkyl group, for example, a straight or branched alkyl chain. In embodiments, at least one of R, R¹, R², R³ may be a methyl, ethyl, propyl, butyl group.

In embodiments wherein R, R¹, R², and/or R³ is an aromatic group, the aromatic group may be one of, or a combination of, an aromatic hydrocarbon group, and/or an aromatic heterocyclic group.

In embodiments wherein R, R¹, R², and/or R³ is an aromatic hydrocarbon group, the aromatic hydrocarbon group may comprise one of, or a combination of, a phenyl ring and/or a substituted phenyl ring. There may be one, two, three, four, or five additional substituents on the phenyl ring. The substituents are bonded directly to the phenyl ring, and may be one of, or a combination of, fluorine, chlorine, bromine, iodine, a hydroxyl group, an amine group, a nitro group, an alkoxy group, a carboxylic acid, an amide, a cyano group, a trifluoromethyl, an ester, an alkene an alkyne, an azide, an azo, an isocyanate, a ketone, an aldehyde, an alkyl group consisting of a hydrocarbon chain, or a hydrocarbon ring, an alkyl group consisting of other heteroatoms such as fluorine, chlorine, bromine, iodine, oxygen, nitrogen, and/or sulphur. The alkyl group may comprise a hydroxyl group, an amine group, a nitro group, an ether group, a carboxylic acid, an amide, a cyano group, trifluoromethyl, an ester, an alkene an alkyne, an azide, an azo, an isocyanate, a ketone, an aldehyde, for example. The substituents may be another aromatic group, for example, R may comprise a phenyl substituted with a further phenyl ring. In embodiments, the R group may be a phenyl ring, substituted with a second phenyl ring, which in turn is substituted with a third phenyl ring. In embodiments, R, R¹, R², or R³ may represent a p-fluorophenyl group, a m-fluorophenyl group, an o-fluorophenyl group, a thiophene group, a cyanophenyl moiety (e.g. a p-cyanophenyl moiety), a trifluoromethylphenyl moiety (e.g. a p-trifluoromethylphenyl moiety), an iodophenyl moiety (e.g. an o-iodophenyl moiety), a chlorophenyl moiety (e.g. an o-chlorophenyl moiety), a bromophenyl moiety (e.g. an o-bromophenyl moiety), an aminophenyl moiety (e.g. a mono-substituted or di-substituted or trisubstituted aminophenyl moiety), a nitrophenyl moiety (e.g. a p-nitrophenyl moiety), a phenol moiety.

In embodiments wherein R, R¹, R², and/or R³ is an aromatic group, the aromatic group may be a polycyclic aromatic hydrocarbon, for example, naphthalene, anthracene, phenanthrene, tetracene, chrysene, triphenylene, pyrene, pentacene, benzo[a]pyrene, corannulene, benzo[ghi]perylene, coronene, ovalene, fullerene, and/or benzo[c]fluorene. The R group may be bonded to the triphenylene derivative by any isomer of the polycyclic aromatic hydrocarbons described, for example, 1-napthalene, 2-napthalene, 2-anthracene, 9-anthracene. The polycyclic aromatic hydrocarbon group may be substituted with other moieties such as aryl groups, alkyl groups, heteroatoms, and/or other electron withdrawing or electron donating groups.

In embodiments wherein R, R¹, R², or R³ is an aromatic heterocyclic group, the heterocyclic group may be a three membered ring, a four membered ring, a five membered ring, a six membered ring, a seven membered ring, an eight membered ring, a nine membered ring, a ten membered ring, or a fused ring. In embodiments, the heterocyclic group may be furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, isoxazole, benzisoxazole, thiazole, benzothiazole, pyridine, quinoline, isoquinoline, pyrazine, quinoxaline, acridine, pyrimidine, quinozoline, pyridazine, cinnoline, phthalazine, 1,2,3-triazine, 1,2,4-triazine, 1,3,5-triazine.pyridine or thiophene.

In embodiments wherein R, R¹, R², or R³ is an aliphatic group, the aliphatic group may be one of, or a combination of, an n-alkyl chain, a branched alkyl chain, an alkyl chain comprising unsaturated moieties, an alkyl chain comprising heteroatoms, for example, fluorine, chlorine, bromine, iodine, oxygen, sulphur, nitrogen. The alkyl chain may comprise unsaturated portions, comprising alkenes, or aromatic moieties. The alkyl chain may comprise functional groups for further derivatisation of the polycyclic aromatic hydrocarbon, e.g. triphenylene, derivative. For example, the functional groups may be one or more of an azide, a carbonyl group, an alcohol, a halogen, an alkene, or a thioacetate.

In embodiments, R, R¹, R², or R³ comprise a crown ether.

The luminescent compound may be any one of the structures shown in FIG. 3.

In some embodiments, the luminescent compound is Compound (2) (TpOx-Ph):

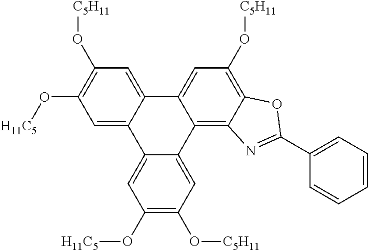

In some embodiments, the luminescent compound is Compound (3) (TpOx-2-Nap):

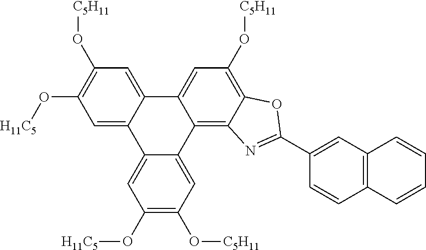

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described by way of example and with reference to the accompanying figures in which:

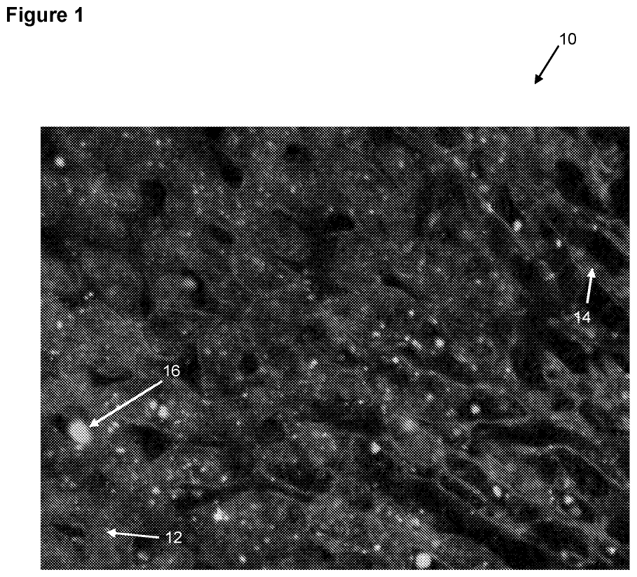

FIG. 1 is a multiphoton microscopy image of an ex vivo of human liver perfused with a composition comprising the fluorophore TpOx-2-Nap;

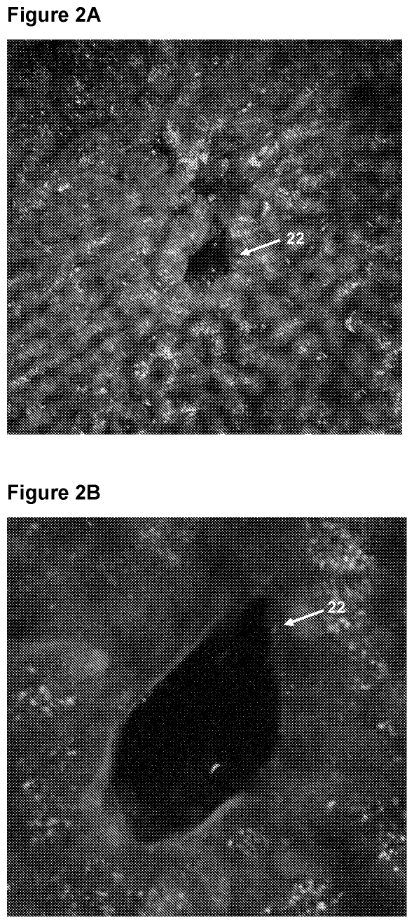

FIG. 2 is a multiphoton microscopy image of an ex vivo of human liver perfused with a composition comprising the fluorophore TpOx-Ph;

FIG. 3 is a table listing the chemical structures of the luminescent compounds that may be used in embodiments of the invention; and

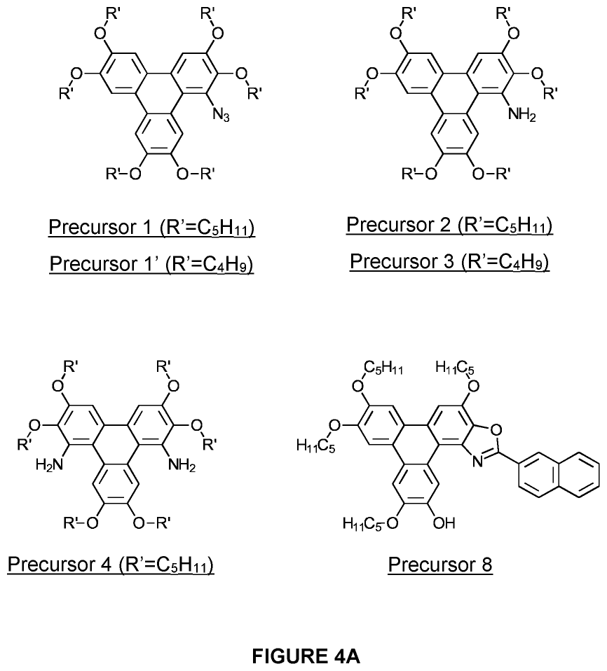

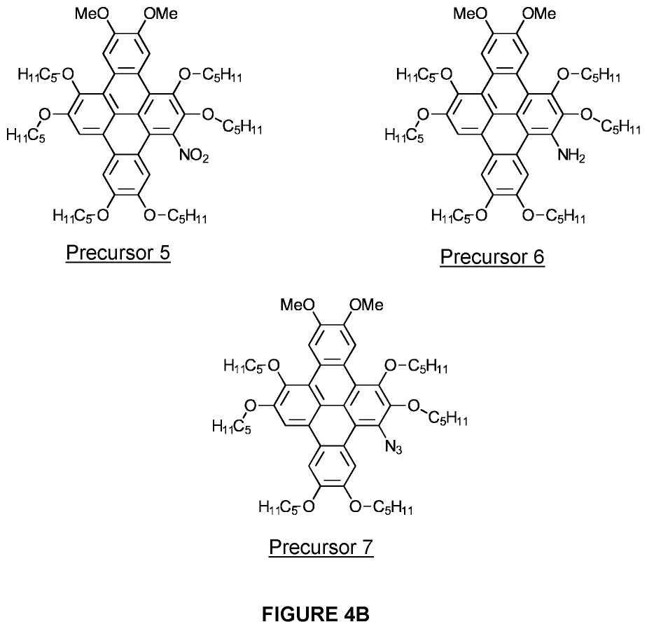

FIGS. 4A and 4B show the structures of the Precursor compounds used to synthesise the luminescent compounds of FIG. 3.

Compounds for use in the invention were synthesised according to the following protocols. All compound names were generated using ChemDraw (RTM) software.

Synthetic Methods

Method of Synthesising Compound 1

Compound 1 was synthesised using the following method from Precursor 1.

Precursor 1 was fabricated according to the method described in N. Boden et. al. J. Mater. Chem., 1995, 5, 2275.

A solution of Precursor 1 (100 mg; 0.13 mmol) in o-xylene (8 mL) was added to a flask. This was then heated and held at 175° C. for 16 h to afford Compound 1 (51% yield).

In the alternative, Compound 1 was synthesised using the following method. A solution of Precursor 1 (100 mg; 0.13 mmol) in dry PhMe (8 mL) was added to a flask containing rhodium octanoate dimer (8 mg; 0.01 mmol), under a N₂ atmosphere. This was then heated and held at reflux for 20 h. The reaction was cooled to room temperature and then evaporated to dryness in vacuo, the solid was then purified via flash column chromatography (silica; 95% n-hexane: 5% ethyl acetate) to afford Compound 1 as a white solid (96 mg, 99%).

The name for Compound 1 is 8-butyl-2,3,6,11,12-pentakis(pentyloxy)triphenyleno[1,2-d]oxazole.

Compound 1 had the following characterisation data: ¹H NMR (300 MHz, CDCl₃) δ_{H}: 10.01 (1 H, s), 7.94 (1H, s), 7.90 (1H, s), 7.88 (1 H, s), 7.85 (1H, s), 4.42 (2H, t, J 6.7 Hz), 4.37 (2H, t, J 6.7 Hz) 4.29-4.23 (6H, m), 3.09 (2H, t, J 7.5 Hz), 2.05-1.92 (10H, m), 1.62-1.43 (24H, m), 1.06-0.96 (18H, m) ppm. ¹³C NMR (100 MHz, CDCl₃) δ_{c}: 165.6, 149.5, 149.1, 148.7, 148.3, 142.9, 140.1, 139.8, 124.6, 123.9, 123.5, 123.3, 116.3, 111.0, 108.3, 106.9, 106.8, 102.6, 69.9, 69.6, 69.5, 68.8, 29.2, 29.0, 28.8, 28.4, 28.3, 22.6, 22.4, 14.2, 13.9 ppm. ES+MS m/z: 756.5 ([M⁺ H]⁺15%), 778.5 ([M⁺ Na]⁺100%). IR λ⁻¹ (neat): 3112w (C—H), 2953m (C—H), 1617w (C═N), 1517w (benzene ring), 1259s (C—O)), 1177s (C—O)), 1159s (C—O)) cm-1. Elemental analysis Found: C, 76.09; H, 9.17; N, 1.95%. C₄₈H₆₉NO₆ requires C, 76.25; H, 9.20; N, 1.85%.

Method of Synthesising Compound 2

Compound 2 was synthesised using the following method from Precursor 2.

Precursor 2 was fabricated according to the method described in N. Boden et. al. J. Mater. Chem., 1995, 5, 2275.

A slurry of benzoic acid (160 mg; 1.31 mmol), palladium diacetate (0.005 mmol) and iodobenzene diacetate (0.16 mmol) in PhMe (5 mL) was heated at 70° C. under N₂ for 20 min. Precursor 2 (100 mg, 0.13 mmol) in PhMe (2 mL) was added and the reaction was heated and held at reflux for 72 h. The mixture was cooled to room temperature and diluted with CH₂Cl₂ (20 mL). The mixture was washed with 1M NaOH (2×20 mL) and the organic phase was dried in vacuo. The crude black solid was purified via flash column chromatography (40% CH₂Cl₂: 60% n-hexane) to afford Compound 2 as a white solid (35 mg, 34%).

The name for Compound 2 is 2,3,6,11,12-pentakis(pentyloxy)-8-phenyltriphenyleno[1,2-d]oxazole.

Compound 2 had the following characterisation data: ¹H NMR (300 MHz, CDCl₃) δ_{H}: 10.13 (1H, s), 8.40-8.37 (2H, m), 7.92 (1H, s), 7.88 (1H, s), 7.87 (1H, s), 7.77 (1H, s), 7.57-7.55 (1H, m), 4.48-4.43 (4H, m), 4.30-4.23 (6H, m), 2.12-1.92 (10H, m), 1.69-1.54 (12H, m), 1.53-1.45 (12H, m), 1.04-0.96 (18H, m) ppm. ¹³C NMR (100 MHz, CDCl₃) δ_{c}: 161.4, 149.5, 149.0, 148.7, 148.3, 142.9, 140.5, 140.2, 131.2, 128.9, 127.5, 127.1, 124.7, 123.8, 123.4, 123.3, 116.4, 110.9, 108.2, 106.8, 106.6, 103.8, 69.8, 69.5, 68.9, 29.2, 29.0, 28.4, 28.3, 22.6, 22.6, 14.1 ppm. ES+MS m/z: 775.5 ([M]⁺22%), 776.5 ([M+H]⁺37%), 798.5 ([M+Na]+100%). Elemental analysis Found: C, 77.46; H, 8.44; N, 1.75%. C₅₀H₆₅NO₆ requires C, 77.38; H, 8.44; N, 1.80%.

Method of Synthesising Compound 3

Compound 3 was synthesised using the following method. A solution of 2-naphthalene carboxylic acid (225 mg, 1.31 mmol), palladium diacetate (0.005 mmol) and iodobenzene diacetate (0.157 mmol) in PhMe (5 mL) was heated at 70° C. under N₂ for 20 min. A solution of Precursor 2 (100 mg; 0.131 mmol) in PhMe (2 mL) was added and heated under reflux for 48-72 h, whilst stirring. The solution was cooled to room temperature and diluted with CH₂Cl₂ (20 mL). The organic phase was washed with aqueous NaOH (1M; 2×20 mL), separated and the organic phase was dried in vacuo. The crude black solid was purified by flash column chromatography (silica; 40% CH₂Cl₂: 60% n-hexane) to afford Compound 3 as a yellow solid (35 mg, 32%).

The name for Compound 3 is 8-(naphthalen-2-yl)-2,3,6,11,12-pentakis(pentyloxy)triphenyleno[1,2-d]oxazole.

Compound 3 had the following characterisation data: ¹H NMR (300 MHz, CDCl₃) δ_{H}: 10.22 (1 H, s), 8.89 (1H, s), 8.49 (1H, dd, J 8.6, 1.7 Hz), 8.05-7.99 (2H, m), 7.96-7.91 (5H, m), 7.62-7.59 (2H, m), 4.54 (2H, t, J 6.8 Hz), 4.51 (2H, t, J 6.8 Hz), 4.32-4.25 (6H, m), 2.17-1.93 (10H, m), 1.76-1.42 (20H, m), 1.06-0.97 (15H, m) ppm. ¹³C NMR (100 MHz, CDCl₃) δ_{c}: 161.7, 149.8, 149.3, 149.0, 148.6, 143.2, 140.9, 140.6, 135.0, 133.4, 129.3, 128.9, 128.3, 128.0, 127.9, 127.4, 127.2, 125.0, 125.0, 124.4, 124.2, 123.7, 123.6, 116.7, 111.2, 108.5, 107.1, 107.0, 103.9, 70.2, 70.1, 69.8, 69.2, 29.6, 29.5, 28.9, 28.8, 28.7, 23.1, 23.0, 14.6, 14.5 ppm. MALDI+m/z: 825.5 ([M]⁺100%). IR λ-1 (neat): Elemental analysis Found: C, 78.95; H, 8.02; N, 1.83%. C₅₄H₈₇NO₆ requires C, 78.51; H, 8.17; N, 1.70%.

Method of Synthesising Compound 4 Compound 4 was synthesised using the following method. A solution of 1-naphthalene carboxylic acid (225 mg, 1.31 mmol), palladium diacetate (0.005 mmol) and iodobenzene diacetate (0.157 mmol) in PhMe (5 mL) was heated at 70° C. under N₂ for 20 min. A solution of Precursor 2 (100 mg; 0.131 mmol) in PhMe (2 mL) was added and heated under reflux for 48-72 h, whilst stirring. The solution was cooled to room temperature and diluted with CH₂Cl₂ (20 mL). The organic phase was washed with aqueous NaOH (1M; 2×20 mL), separated and the organic phase was dried in vacuo. The crude black solid was purified by flash column chromatography (silica; 40% CH₂Cl₂: 60% n-hexane) to afford Compound 4 as a yellow solid (24 mg, 22%).

The name for Compound 4 is 8-(naphthalen-1-yl)-2,3,6,11,12-pentakis(pentyloxy)triphenyleno[1,2-d]oxazole.

Compound 4 had the following characterisation data: ¹H NMR (300 MHz, CDCl₃) δ_{H}: 10.15 (1H, s), 9.82 (1H, d, J 8.3 Hz), 8.59 (1H, dd, J 7.3, 1.2 Hz), 8.08 (1H, d, J 8.3 Hz), 8.01-7.98 (3H, m), 7.94 (2H, m), 7.71-7.61 (3H, m), 4.54-4.45 (4H, m), 4.32-4.26 (6H, m), 2.10-1.94 (10H, m), 1.70-1.35 (20H, m), 1.04-0.87 (15H, m) ppm. ¹³C NMR (100 MHz, CDCl₃) δ_{c}: 161.3, 149.9, 149.6, 149.1, 148.8, 143.2, 141.0, 139.9, 134.5, 132.4, 131.0, 129.6, 129.2, 127.8, 127.5, 126.9, 126.7, 125.5, 125.0, 124.2, 124.1, 123.8, 117.0, 111.0, 108.6, 107.3, 107.2, 104.4, 70.2, 69.9, 69.1, 29.6, 29.5, 29.0, 28.8, 28.7, 23.0, 14.5 ppm. MALDI+m/z: 826.7 ([M+H]⁺ 100%). Elemental analysis Found: C, 78.49; H, 8.23; N, 1.73%. C₅₄H₆₇NO₆ requires C, 78.51; H, 8.17; N, 1.70%.

Method of Synthesising Compound 5

Compound 5 was synthesised using the following method. A solution of 2-anthracene carboxylic acid (290 mg, 1.31 mmol), palladium diacetate (0.005 mmol) and iodobenzene diacetate (0.157 mmol) in PhMe (5 mL) was heated at 70° C. under N₂ for 20 min. A solution of Precursor 2 (100 mg; 0.131 mmol) in PhMe (2 mL) was added and heated under reflux for 48-72 h, whilst stirring. The solution was cooled to room temperature and diluted with CH₂Cl₂ (20 mL). The organic phase was washed with aqueous NaOH (1M; 2×20 mL), separated and the organic phase was dried in vacuo. The crude black solid was purified by flash column chromatography (silica; 40% CH₂Cl₂: 60% n-hexane) to afford Compound 5 as a yellow solid (22 mg, 20%).

The name for Compound 5 is 8-(anthracen-2-yl)-2,3,6,11,12-pentakis(pentyloxy)triphenyleno[1,2-d]oxazole.

Compound 5 had the following characterisation data: ¹H NMR (300 MHz, CDCl₃) δ_{H}: 10.20 (1H, s), 9.00 (1H, s), 8.58 (1H, s), 8.47 (1H, s), 8.39 (1H, dd, J 8.9, 1.6 Hz), 8.13-8.10 (2H, m), 8.07-8.02 (2H, m), 7.93 (1H, s), 7.90-7.89 (3H, m), 4.57-4.47 (4H, m), 4.31-4.24 (6H, m), 2.19-1.96 (10H, m), 1.76, 1.44 (20H, m), 1.08-0.97 (15H, m) ppm. ¹³C NMR (100 MHz, CDCl₃) δ_{c}: 161.8, 149.8, 149.4, 149.0, 148.6, 143.2, 141.0, 140.6, 133.1, 132.6, 132.3, 131.2, 129.2, 128.7, 128.6, 128.2 127.4, 126.8, 126.6, 126.3, 125.0, 124.4, 124.2, 123.8, 123.6, 116.7, 111.3, 108.5, 107.1, 107.0, 104.0, 70.2, 70.1, 69.8, 69.2, 30.1, 29.6, 29.5, 28.9, 28.8, 28.7, 23.1, 23.0, 14.7, 14.5 ppm. MALDI+m/z: 876.5 ([M+H]⁺100%). Elemental analysis Found: C, 79.49; H, 7.88; N, 1.51%. C₅₈H₆₉NO₆ requires C, 79.51; H, 7.94; N, 1.60%.

Method of Synthesising Compound 6

Compound 6 was synthesised using the following method. A solution of 9-anthracene carboxylic acid (290 mg; 1.31 mmol), palladium diacetate (0.005 mmol) and iodobenzene diacetate (0.157 mmol) in o-xylene (5 mL) was heated to 70° C. under N₂ for 20 min. A solution of Precursor 2 (100 mg, 0.131 mmol) in o-xylene (2 mL) was added and heated to 140° C. for 72 h. The mixture was cooled to room temperature and diluted with CH₂Cl₂ (20 mL). The mixture was washed with 1M NaOH (2×20 mL) and the organic phase was dried in vacuo. The crude black solid was purified by flash column chromatography (silica; 40% CH₂Cl₂: 60% n-hexane) to afford Compound 6 as a yellow solid (13 mg, 11%).

The name for Compound 6 is 8-(anthracen-9-yl)-2,3,6,11,12-pentakis(pentyloxy)triphenyleno[1,2-d]oxazole.

Compound 6 had the following characterisation data: ¹H NMR (300 MHz, CDCl₃) δ_{H}: 10.18 (1 H, s), 8.70 (1H, s), 8.49-8.44 (2H, m), 8.15-8.09 (2H, m), 8.03 (1H, s), 8.02 (1H, s) 7.95 (1H, s), 7.94 (1H, s) 7.58-7.52 (4H, m), 4.50 (2H, t, J 6.7 Hz), 4.33-4.27 (6H, m), 4.17 (2 H, t, J 6.7 Hz), 2.05-1.93 (8H, m), 1.79 (2H, p, J 6.7, 1.0 Hz), 1.66-1.37 (20H, m), 1.03-0.92 (15H, m) ppm. ¹³C NMR δ_{c}: (100 MHz, CDCl₃) 160.6, 150.0, 149.6, 149.2, 148.8, 143.5, 141.0, 140.7, 131.9, 131.7, 131.3, 129.1, 127.7, 127.6, 126.4, 125.9, 124.2, 123.8, 121.1, 117.1, 111.3, 108.7, 107.3, 107.2, 104.6, 70.4, 70.3, 69.9, 69.2, 29.6, 29.5, 29.0, 28.8, 28.7, 28.6, 28.5, 23.0, 22.9, 22.6, 14.5, 14.4, 14.3 ppm. MALDI⁺ m/z: 876.5 ([M+H]⁺ 100%). Elemental analysis Found: C, 79.13; H, 7.83; N, 1.77%. C₅₈H₆₉NO₆ requires C, 79.51; H, 7.94; N, 1.60%.

Method of Synthesising Compound 7

Compound 7 was synthesised using the following method. A solution of 4-fluorobenzoic acid (187 mg; 1.31 mmol), palladium diacetate (0.005 mmol) and iodobenzene diacetate (0.157 mmol) in o-xylene (5 mL) was heated to 70° C. under N₂ for 20 min. A solution of Precursor 2 (100 mg, 0.131 mmol) in o-xylene (2 mL) was added and heated to 140° C. for 72 h. The mixture was cooled to room temperature and diluted with CH₂Cl₂ (20 mL). The mixture was washed with 1M NaOH (2×20 mL) and the organic phase was dried in vacuo. The crude black solid was purified by flash column chromatography (silica; 40% CH₂Cl₂: 60% n-hexane) to afford Compound 7 as a yellow solid (13 mg, 9%).

The name for Compound 7 is 8-(4-fluorophenyl)-2,3,6,11,12-pentakis(pentyloxy)triphenyleno[1,2-d]oxazole.

Compound 7 had the following characterisation data: ¹H NMR (300 MHz; CDCl₃) δ_{H}: 10.03 (1H, s), 8.36-8.30 (2H, m), 7.97-7.75 (4H, m), 7.28-7.15 (3H, m), 4.41 (4H, t, J 6.6 Hz), 4.26 (6H, m), 2.06-1.90 (9H, m), 1.55 (22H, m), 1.05-0.95 (15H, m) ppm. ¹³C NMR (100 MHz; CDCl₃) δ_{c}: 166.0, 163.5, 160.6, 149.7, 149.1, 148.9, 148.5, 143.0, 140.5, 140.3, 129.8, 129.7, 127.3, 124.8, 123.9, 123.8, 123.5, 116.5, 116.3, 116.1, 111.1, 108.4, 107.0, 106.8, 103.7, 69.9, 69.8, 69.7, 69.5, 68.9, 29.7, 29.2, 29.0, 28.5, 28.4, 28.3, 22.6, 14.2, 14.1 ppm. ¹⁹F NMR (282 MHz, CDCl₃) δ_{F}: −108.0 ppm. MALDI+m/z: 793.6 ([M]⁺ 100%), 794.6 ([M+H]⁺ 55%), 795.6 ([M+H+1]⁺15%). IR λ⁻¹ (neat): 2952m (C—H), 2926m (C—H), 2858m (C—H), 1616w (C═N), 1517s (benzene ring), 1499m (benzene ring), 1433m (benzene ring), 1261m (C—O)), 1174s (C—O)) cm⁻¹.

Method of Synthesising Compound 8

Compound 8 was synthesised using the following method. A solution of 3-fluorobenzoic acid (182 mg; 1.30 mmol), palladium diacetate (0.005 mmol) and iodobenzene diacetate (0.157 mmol) in o-xylene (5 mL) was heated to 70° C. under N₂ for 20 min. A solution of Precursor 2 (100 mg, 0.131 mmol) in o-xylene (2 mL) was added and heated to 140° C. for 72 h. The mixture was cooled to room temperature and diluted with CH₂Cl₂ (20 mL). The mixture was washed with 1M NaOH (2×20 mL) and the organic phase was dried in vacuo. The crude black solid was purified by flash column chromatography (silica; 40% CH₂Cl₂: 60% n-hexane) to afford Compound 8 as a yellow solid (13 mg, 11%).

The name for Compound 8 is 8-(3-fluorophenyl)-2,3,6,11,12-pentakis(pentyloxy)triphenyleno[1,2-d]oxazole.

Compound 8 had the following characterisation data: ¹H NMR (300 MHz; CDCl₃) δ_{H}: 10.06 (1H, s), 8.15 (1H, d, J 7.9 Hz), 8.05 (1H, dd, J 9.0, 1.9 Hz, 7.89 (4H, m), 7.53 (m, 1H), 7.36-7.16 (4H, m), 4.45 (4H, m), 4.33-4.21 (6H, m), 2.16-1.90 (11H, m), 1.71-1.39 (24H, m), 1.06-0.93 (15H, m ppm. ¹³C NMR (100 MHz; CDCl₃) δ_{c}: 164.3, 161.8, 160.1, 160.1, 149.6, 149.1, 148.8, 148.4, 142.9, 140.3, 140.3, 130.6, 130.5, 129.6, 129.6, 127.3, 124.8, 123.7, 123.4, 123.4, 123.2, 123.2, 118.3, 118.1, 116.4, 114.6, 114.3, 110.9, 108.2, 106.8, 106.7, 103.9, 69.9, 69.8, 69.6, 69.0, 29.4, 29.3, 29.2, 28.6, 28.5, 28.4, 22.8, 14.3 ppm. 19F NMR (282 MHz; CDCl₃) δ_{F}: −111.8 ppm. ES+MS m/z: 794.5 ([M]⁺ 55%), 816.5 ([M+Na]⁺100%), 817.5 ([M+H*Na]⁺50%). IR λ-1 (neat): 2952m (C—H), 2925m (C—H), 2856m (C—H), 1617w (C═N), 1518s (benzene ring), 1434s (benzene ring), 1262s (C—O), 1174s (C—O)) cm-1. Elemental analysis Found: C, 75.62; H, 8.25; N, 1.78%. C₅₀H₆₄FNO₆ requires C, 75.63; H, 8.12; N, 1.76%.

Method of Synthesising Compound 9

Compound 9 was synthesised using the following method. A solution of 2-fluorobenzoic acid (41.86 mg; 0.26 mmol), palladium diacetate (0.005 mmol) and iodobenzene diacetate (0.157 mmol) in o-xylene (5 mL) was heated to 70° C. under N₂ for 20 min. A solution of Precursor 2 (100 mg, 0.131 mmol) in o-xylene (2 mL) was added and heated to 140° C. for 72 h. The mixture was cooled to room temperature and diluted with CH₂Cl₂ (20 mL). The mixture was washed with 1M NaOH (2×20 mL) and the organic phase was dried in vacuo. The crude black solid was purified by flash column chromatography (silica; 40% CH₂Cl₂: 60% n-hexane) to afford Compound 9 as a yellow solid (7 mg, 10%).

The name for Compound 9 is 8-(2-fluorophenyl)-2,3,6,11,12-pentakis(pentyloxy)triphenyleno[1,2-d]oxazole.

Compound 9 had the following characterisation data: ¹H NMR (300 MHz; CDCl₃) δ_{H}: 10.16 (1H, s), 8.38 (1H, m), 7.92 (4H, m), 7.63-7.47 (1H, m), 7.43-7.28 (2H, m), 4.47 (4H, m), 4.27 (5H, m), 2.13-1.91 (9H, m), 1.69-1.39 (21H, m), 1.00 (14H, m) ppm. 13C NMR (100 MHz; CDCl₃) δ_{c}: 162.4, 159.8, 157.6, 157.5, 149.7, 149.3, 148.8, 148.4, 142.9, 140.6, 140.5, 139.9, 132.9, 132.8, 130.3, 127.3, 124.8, 124.5, 123.9, 123.4, 123.4, 117.4, 117.2, 116.7, 116.0, 115.9, 110.9, 108.3, 107.0, 106.9, 104.4, 69.8, 69.5, 68.9, 29.2, 29.0, 28.4, 28.3, 22.6, 22.6, 14.1 ppm. 19F NMR (282 MHz; CDCl₃) δ_{F}: −109.1 ppm. MALDI+m/z: 793.6 ([M]⁺ 100%), 794.6 ([M+H]⁺ 65%), 795.6 ([M+1+H]⁺ 20%). IR λ-1 (neat): 2952m (C—H), 2925m (C— H), 2856m (C—H), 1617w (C═N), 1518m (benzene ring), 1434m (benzene ring), 1261s (C—O)), 1176s (C—O)) cm-1. Elemental analysis Found: C, 75.92; H, 8.26; N, 1.74%. C₅₀H₆₄FNO₆ requires C, 75.63; H, 8.12; N, 1.76%.

Method of Synthesising Compound 10

Compound 10 was synthesised using the following method. A slurry of Precursor 2 (100 mg; 0.01 mmol), iodobenzene diacetate (51 mg; 0.16 mmol) and palladium diacetate (1 mg; 0.005 mmol) in a mixture of PhMe (5 mL) and acetic acid (1 mL) in PhMe (5 mL) under an N₂ atmosphere was heated and held at reflux for 72 h. The reaction was then cooled to room temperature and washed with 1M NaOH (1M; 2×10 mL). The organic phase was evaporated to dryness in vacuo. The solid was then purified via flash column chromatography (40% CH₂Cl₂: 60% n-hexane) to afford Compound 10 as a white solid (64 mg; 66%).

The name for Compound 10 is 8-methyl-2,3,6,11,12-pentakis(pentyloxy)triphenyleno[1,2-d]oxazole.

Compound 10 had the following characterisation data: ¹H NMR δ_{H}: (400 MHz, CDCl₃) 9.94 (1H, s), 7.94 (1H, s), 7.90 (1H, s), 7.89 (1H, s), 7.85 (1H, s), 4.42 (2H, t, J 6.7 Hz), 4.38 (2H, t, J 6.8), 4.30-4.24 (6H, m), 2.81 (3H, s), 1.99 (10H, m), 1.65-1.53 (10H, m), 1.52-1.44 (10H, m), 1.03-0.96 (15H, m) ppm. ¹³C NMR δ_{c}: (100 MHz, CDCl₃) 162.4, 149.9, 149.4, 149.1, 148.8, 143.2, 140.8, 140.2, 127.2, 125.0, 124.2, 123.9, 123.7, 116.7, 111.6, 108.8, 107.3, 107.2, 102.9, 70.3, 70.2, 69.88, 69.40, 29.60, 29.5, 29.3, 28.8, 28.8, 28.6, 23.0, 15.2, 14.5 ppm. MALDI m/z: 714.5 ([M]⁺ 100%).

Method of Synthesising Compound 11

Compound 11 was synthesised using the following method. A solution of Precursor 2 (200 mg, 0.263 mmol) and trimethylamine (0.2 mL, 1.44 mmol) in PhMe (7 mL) was heated at reflux under N₂ for 10 min. 2-Thiophenecarbonyl chloride (0.3 mL, 2.62 mmol) was added and heated under reflux for 90 min. The solution was cooled to room temperature and washed with 1M HCl (30 mL) and the organic phase extracted with EtOAc (2×30 mL). The organic phase was dried in vacuo and the resultant black solid was heated at 240° C. for 10 min before being cooled to room temperature. The crude 55 black solid was purified by flash column chromatography (silica; 40% CH₂Cl₂: 60% n-hexane) to afford Compound 11 as a yellow solid (136 mg; 64%).

The name for Compound 11 is 2,3,6,11,12-pentakis(pentyloxy)-8-(thiophen-2-yl)triphenyleno[1,2-d]oxazole.

Compound 11 had the following characterisation data: ¹H NMR δ_{H}: (300 MHz, CDCl₃) 10.03 (1H, s), 7.99 (1H, dd, J 3.7, 1.2 Hz), 7.92 (1H, s), 7.89 (1H, s), 7.88 (1H, s), 7.88 (1H, s), 7.58 (1H, dd, J 5.0, 1.2), 7.25-7.22 (1H, dd, J 5.0, 3.7 Hz), 4.48-4.43 (4H, m), 4.31-4.23 (6H, m), 2.14-1.93 (10H, m), 1.71-1.42 (20H, m), 1.04-0.97 (15H, m) ppm. ¹³C NMR δ_{c}: (100 MHz, CDCl₃) 157.8, 149.8, 149.4, 149.0, 148.6, 143.0, 140.7, 140.1, 130.6, 130.1, 129.7, 128.5, 127.5, 125.0, 124.1, 123.7, 123.6, 116.6, 111.1, 108.5, 107.1, 107.1, 104.0, 70.2, 70.2, 70.1, 69.8, 69.1, 29.6, 29.5, 29.4, 28.9, 28.8, 28.8, 28.6, 23.0, 23.0, 14.6, 14.5 ppm. MALDI m/z: 781.5 ([M]⁺ 100%).

Method of Synthesising Compound 12

Compound 12 was synthesised using the following method. A solution of Precursor 2 (100 mg, 0.132 mmol), 4-Cyanobenzoyl chloride (109 mg, 0.658 mmol) and N,N-Diisopropylethylamine (0.1 mL, 0.574 mmol) in PhMe (5 mL) was heated to and held at reflux for 18 h under N₂. The reaction was cooled to room temperature and then evaporated to dryness in vacuo. The solid was then heated and held at 240° C. for 15 min under N₂. The crude black solid was then cooled to room temperature and purified via flash column chromatography (silica, 40% CH₂Cl₂: 60% n-hexane) to afford Compound 12 as a yellow solid (40 mg, 38%).

The name for Compound 12 is 4-(2,3,6,11,12-pentakis(pentyloxy)triphenyleno[1,2-d]oxazol-8-yl)benzonitrile.

Compound 12 had the following characterisation data: ¹H NMR δH: (300 MHz, CDCl₃) 9.96 (1H, s), 8.43-8.40 (2H, d, J 8.55 Hz), 7.90 (1H, s), 7.89 (1H, s), 7.88 (1H, s), 7.87 (1H, s), 7.83-7.80 (2H, d, J 8.55), 4.43-4.38 (4H, m), 4.30-4.23 (6H, m), 2.12-1.93 (10H, m), 1.67-1.42 (20H, m), 1.04-0.97 (15H, m) ppm. ¹³C NMR δ_{c}: (100 MHz, CDCl₃) 159.3, 150.0, 149.3, 149.1, 148.8, 143.1, 140.6, 140.4, 132.7, 131.4, 127.8, 127.6, 125.0, 123.7, 123.7, 123.4, 118.7, 116.6, 114.4, 111.0, 108.4, 106.9, 106.7, 104.4, 70.2, 70.0, 69.9, 69.8, 69.2, 29.6, 29.5, 29.4, 28.8, 28.8, 28.8, 28.7, 23.0, 14.6, 14.5 ppm. MALDI m/z: 800.4 ([M]⁺ 100%).

Method of Synthesising Compound 13

Compound 13 was synthesised using the following method. A solution of 4-(trifluoromethyl)benzoic acid carboxylic acid (248 mg, 1.31 mmol), palladium diacetate (0.005 mmol) and iodobenzene diacetate (0.157 mmol) in PhMe (5 mL) was heated at 60° C. under N₂ for 30 min. A solution of Precursor 2 (100 mg, 0.132 mmol) in PhMe (2 mL) was added and heated under reflux for 48-72 h, whilst stirring. The solution was cooled to room temperature and diluted with CH₂Cl₂ (20 mL). The organic phase was washed with aqueous NaOH (1M; 2×20 mL), separated and the organic phase was dried in vacuo. The crude black solid was purified by flash column chromatography (silica; 40% CH₂Cl₂: 60% n-hexane) to afford Compound 13 as a yellow solid (6 mg; 5%).

The name for Compound 13 is 2,3,6,11,12-pentakis(pentyloxy)-8-(4-(trifluoromethyl)phenyl)triphenyleno[1,2-d]oxazole.

Compound 13 had the following characterisation data: ¹H NMR δ_{H}: (300 MHz, CDCl₃) 10.04 (1H, s), 8.48-8.46 (2H, d, J 8.50), 7.92 (1H, s), 7.90 (1H, s), 7.89 (1H, s), 7.88 (1H, s), 7.83-7.80 (2H, d, J 8.50), 4.47-4.45 (4H, t, J 6.74), 4.31-4.24 (6H, m), 2.14-1.94 (10H, m), 1.70-1.43 (20H, m), 1.05-0.98 (15H, m) ppm. ¹⁹F NMR δ_{F}: (300 MHz, CDCl₃) 62.9 (s) ppm. ¹³C NMR δ_{c}: (100 MHz, CDCl₃) 159.3, 150.0, 149.3, 149.1, 148.8, 143.1, 140.6, 140.4, 132.7, 131.4, 127.8, 127.6, 125.0, 123.7, 123.7, 123.4, 118.7, 116.6, 114.4, 111.0, 108.4, 106.9, 106.7, 104.4, 70.2, 70.0, 69.9, 69.8, 69.2, 29.6, 29.5, 29.4, 28.8, 28.8, 28.8, 28.7, 23.0, 14.6, 14.5 ppm. MALDI m/z: 844.5 ([M+H]⁺ 100%).

Method of Synthesising Compound 14

Compound 14 was synthesised using the following method. A solution of Precursor 2 (100 mg, 0.132 mmol), 2-iodobenzoyl chloride (175 mg, 0.658 mmol) and N,N-Diisopropylethylamine (0.1 mL, 0.574 mmol) in PhMe (5 mL) was heated to and held at reflux for 18 h under N₂. The reaction was cooled to room temperature and then evaporated to dryness in vacuo. The solid was then heated and held at 240° C. for 15 min under N₂. The crude black solid was then cooled to room temperature and purified via flash column chromatography (silica, 40% CH₂Cl₂: 60% n-hexane) to afford Compound 14 as a yellow solid (39.9 mg, 35%).

The name for Compound 14 is 8-(2-iodophenyl)-2,3,6,11,12-pentakis(pentyloxy)triphenyleno[1,2-d]oxazole.

Compound 14 had the following characterisation data: ¹H NMR δH: (300 MHz, CDCl₃) 10.07 (1H, s), 8.20-8.16 (1H, dd, J 7.90, 1.60 Hz), 8.18-8.15 (1H, dd, J 7.90, 1.25 Hz), 7.95 (1H, s), 7.94 (1H, s), 7.91 (2H, m), 7.58-7.53 (1H, td, J 7.66, 7.63, 1.25 Hz), 7.26-7.20 (1H, td, J 7.66, 7.63, 1.60 Hz), 4.52-4.44 (4H, m), 4.31-4.25 (6H, m), 2.06-1.94 (10H, m), 1.67-1.41 (20H, m), 1.03-0.92 (15H, m) ppm. ¹³C NMR δ_{c}: (100 MHz, CDCl₃) 160.9, 149.9, 149.6, 149.2, 149.1, 143.3, 142.0, 140.6, 140.4, 132.5, 132.1, 132.1, 128.5, 127.6, 125.1, 124.1, 124.0, 123.7, 117.2, 111.9, 108.6, 107.3, 107.1, 104.9, 95.0, 70.4, 70.2, 70.2, 70.1, 69.9, 29.6, 29.6, 29.5, 29.4, 28.9, 28.8, 28.7, 23.0, 23.0, 14.5 ppm. MALDI m/z: 901.6 ([M]⁺ 14%).

Method of Synthesising Compound 15

Compound 15 was synthesised using the following method. A solution of Precursor 2 (100 mg, 0.132 mmol), 2-chlorobenzoyl chloride (175 mg, 0.658 mmol) and N,N-Diisopropylethylamine (0.1 mL, 0.574 mmol) in PhMe (5 mL) was heated to and held at reflux for 18 h under N₂. The reaction was cooled to room temperature and then evaporated to dryness in vacuo. The solid was then heated and held at 240° C. for 15 min under N₂. The crude black solid was then cooled to room temperature and purified via flash column chromatography (silica, 40% CH₂Cl₂: 60% n-hexane) to afford Compound 15 as a yellow solid (52.4 mg, 49%).

The name for Compound 15 is 8-(2-chlorophenyl)-2,3,6,11,12-pentakis(pentyloxy)triphenyleno[1,2-d]oxazole.

Compound 15 had the following characterisation data: ¹H NMR δ_{H}: (300 MHz, CDCl₃) 10.06 (1H, s), 8.39-8.34 (1H, m), 7.92-7.90 (4H, m), 7.66 (1H, m), 7.51-7.44 (2H, m), 4.49-4.41 (4H, m), 4.30-4.25 (6H, m), 2.06-1.95 (10H, m), 1.67-1.43 (20H, m), 1.03-0.92 (15H, m) ppm. ¹³C NMR δ_{c}: (100 MHz, CDCl₃) 158.8, 149.6, 149.4, 148.9, 148.5, 142.9, 140.4, 139.9, 133.5, 131.7, 131.6, 127.3, 127.0, 126.2, 124.8, 123.9, 123.4, 116.8, 110.9, 108.2, 106.9, 104.6, 70.1, 70.0, 69.9, 69.6, 69.1, 29.8, 29.3, 29.3, 29.2, 28.5, 28.4, 22.7, 14.3 ppm. MALDI m/z: 809.7 ([M]⁺ 95%).

Method of Synthesising Compound 16

Compound 16 was synthesised using the following method. A solution of Precursor 2 (100 mg, 0.132 mmol), 2-bromobenzoyl chloride (144 mg, 0.658 mmol) and N,N-Diisopropylethylamine (0.1 mL, 0.574 mmol) in PhMe (5 mL) was heated to and held at reflux for 18 h under N₂. The reaction was cooled to room temperature and then evaporated to dryness in vacuo. The solid was then heated and held at 240° C. for 15 min under N₂. The crude black solid was then cooled to room temperature and purified via flash column chromatography (silica, 40% CH₂Cl₂: 60% n-hexane) to afford Compound 16 as a yellow solid (25.8 mg, 21%).

The name for Compound 16 is 8-(2-bromophenyl)-2,3,6,11,12-pentakis(pentyloxy)triphenyleno[1,2-d]oxazole.

Compound 16 had the following characterisation data: ¹H NMR δH: (300 MHz, CDCl₃) 10.03 (1H, s), 8.31-8.28 (1H, dd, J 7.91, 1.75 Hz), 7.91-7.89 (4H, m), 7.86-7.83 (1H, dd, J 7.91, 1.23 Hz), 7.54-7.49 (1H, td, J 7.70, 7.60, 1.23 Hz), 7.42-7.37 (1H, td, J 7.70, 7.57, 1.75 Hz) 4.49-4.39 (4H, m), 4.31-4.23 (6H, m), 2.06-1.95 (10H, m), 1.64-1.43 (20H, m), 1.03-0.95 (15H, m) ppm. ¹³C NMR δ_{c}: (100 MHz, CDCl₃): 159.5, 149.7, 149.4, 148.9, 148.6, 142.9, 140.3, 135.0, 132.2, 131.8, 128.3, 127.6, 127.4, 124.8, 123.9, 123.6, 123.5, 122.0, 116.9, 111.1, 108.2, 107.0, 104.7, 70.1, 70.0, 69.9, 69.6, 69.4, 29.3, 29.3, 29.2, 28.6, 28.5, 28.4, 22.7, 14.3 ppm. MALDI m/z: 855.7 ([M]⁺ 31%)

Method of Synthesisinq Compound 17

Compound 17 was synthesised using the following method. A solution of 5-bromovaleric acid (773 mg, 4.27 mmol), palladium diacetate (0.005 mmol) and iodobenzene diacetate (0.512 mmol) in PhMe (10 mL) was heated at 70° C. under N₂ for 20 min. A solution of Precursor 2 (325 mg; 0.428 mmol) in PhMe (10 mL) was added and heated under reflux for 48-72 h, whilst stirring. The solution was cooled to room temperature and diluted with CH₂Cl₂ (20 mL). The organic phase was washed with aqueous NaOH (1M; 2×20 mL), separated and the organic phase was dried in vacuo. The crude black solid was purified by flash column chromatography (silica; 40% CH₂Cl₂: 60% n-hexane) to afford Compound 17 as a white solid (109 mg, 31%).

The name for Compound 17 is 8-(4-bromobutyl)-2,3,6,11,12-pentakis(pentyloxy)triphenyleno[1,2-d]oxazole.

Compound 17 had the following characterisation data: ¹H NMR δ_{H}: (300 MHz, CDCl₃) 9.95 (1H, s), 7.92 (1H, s), 7.90 (1H, s), 7.89 (1H, s), 7.84 (1H, s), 4.43-4.34 (4H, m), 4.30-4.24 (6H, m), 3.55-3.50 (2H, t, J 6.29 Hz), 3.15-3.10 (2H, t, J 7.05 Hz), 2.35 (3H, s), 2.25-1.94 (12H, m), 1.64-1.45 (24H, m), 1.03-0.97 (15H, m) ppm. ¹³C NMR δ_{c}: (100 MHz, CDCl₃) 164.6, 149.6, 149.1, 148.8, 148.4, 142.9, 140.1, 139.7, 127.0, 124.7, 123.8, 123.5, 123.4, 116.3, 111.0, 108.4, 107.0, 102.7, 70.0, 69.9, 69.6, 69.6, 68.9, 33.0, 31.9, 29.3, 29.2, 29.2, 29.1, 28.6, 28.5, 28.4, 28.3, 22.7, 25.3, 22.7, 22.6, 14.3, 14.2, 14.1 ppm. ES+m/z: 834.4 ([M+H]⁺ 95%), 836.4 ([M+H]⁺ 100%).

Method of Synthesisinq Compound 18

Compound 18 was synthesised using the following method. A solution of Compound 17 (66 mg, 0.079 mmol) in acetone (10 mL) was heated to 50° C. and stirred under N₂, to this was added a solution of sodium azide (7 mg, 0.111 mmol) in H₂O (5 mL) and left stirring under N₂ for 4 h. After this time a precipitate had formed and the solvent was removed under reduced pressure, the precipitate was then filtered under vacuum and dried to give Compound 18 as an off white solid (59 mg, 94%).

The name for Compound 18 is 8-(4-azidobutyl)-2,3,6,11,12-pentakis(pentyloxy)triphenyleno[1,2-d]oxazole.

Compound 18 had the following characterisation data: ¹H NMR δ_{H}: (300 MHz, CDCl₃) 9.96 (1H, s), 7.94 (1H, s), 7.90 (1H, s), 7.90 (1H, s), 7.87 (1H, s), 4.45-4.40 (2H, t, J 6.70 Hz), 4.39-4.35 (2H, t, J 6.67 Hz), 4.30-4.24 (6H, m), 3.55-3.51 (2H, t, J 6.31 Hz), 3.17-3.12 (2H, t, J 7.11 Hz), 2.28-1.93 (12H, m), 1.65-1.41 (24H, m), 1.03-0.97 (15H, m) ppm. ¹³C NMR δ_{c}: (100 MHz, CDCl₃) 165.0, 149.6, 149.9, 149.4, 149.1, 148.7, 143.2, 140.4, 140.0, 127.3, 125.0, 124.1, 123.8, 123.7, 116.7, 111.3, 108.7, 107.3, 103.0, 70.3, 70.3, 69.9, 69.9, 69.2, 33.3, 32.3, 29.6, 29.5, 29.5, 29.4, 28.9, 28.8, 28.7, 28.6, 28.0, 25.6, 23.1, 23.0, 14.6, 14.5, 14.5 ppm. ES+m/z: 819.5 ([M+Na]⁺100%).

Method of Synthesisinq Compound 19

Compound 19 was synthesised using the following method. Compound 17 (21 mg, 0.025 mmol) was dissolved in anhydrous THF (4 mL) to this mixture potassium thioacetate (12 mg, 0.1 mmol) was added and stirred under N₂ for 6 h. The organic phase was then extracted with DCM (10 mL) and washed with water (2×10 mL). The organic phase was then dried in vacuo and the solid recrystalised with DCM:MeOH (1 mL: 5 mL). The resultant precipitate was filtered under suction and the solid washed with methanol to give Compound 19 as an off white solid (4 mg, 19%).

The name for Compound 19 is S-(4-(2,3,6,11,12-pentakis(pentyloxy)triphenyleno[1,2-d]oxazol-8-yl)butyl) ethanethioate.

Compound 19 had the following characterisation data: ¹H NMR δ_{H}: (300 MHz, CDCl₃) 9.97 (1H, s), 7.94 (1H, s), 7.90 (1H, s), 7.89 (1H, s), 7.86 (1H, s), 4.45-4.35 (4H, m), 4.30-4.24 (6H, m), 3.14-3.09 (2H, t, J 7.44 Hz), 3.02-2.97 (2H, t, J 7.21 Hz), 2.35 (3H, s), 2.18-1.80 (12H, m), 1.65-1.42 (24H, m), 1.03-0.97 (15H, m) ppm. ¹³C NMR δ_{c}: (100 MHz, CDCl₃) 196.0, 165.2, 149.9, 149.4, 149.1, 148.7, 143.2, 140.4, 140.0, 127.2, 125.0, 124.2, 123.8, 123.6, 116.7, 111.4, 108.7, 107.3, 103.1, 70.3, 70.2, 69.9, 69.9, 69.2, 31.0, 30.1, 29.6, 29.5, 29.5, 29.4, 29.4, 29.1, 28.9, 28.8, 28.7, 28.6, 28.5, 26.2, 23.0, 23.0, 14.6, 14.5, 14.5, 14.5 ppm. MALDI m/z: 829.5 ([M]⁺ 100%).

Method of Synthesisinq Compound 20

Compound 22 was synthesised using the following method. A solution of Compound 17 (260 mg, 0.311 mmol), Sodium Tert-butoxide (90 mg, 0.934 mmol), Potassium Iodide (40 mg, 0.311 mmol) and Ethylene Glycol (193 mg, 3.11 mmol) in MeCN (15 mL) was heated to and held at reflux for 48 h under N₂. The reaction was cooled to room temperature and then evaporated to dryness in vacuo. The crude solid was dissolved with CH₂Cl₂ (20 mL). The organic phase was washed with aqueous NaOH (1M; 2×20 mL) and then HCl (1M, 2×20 mL), separated and the organic phase was dried in vacuo. The crude black solid was then cooled to room temperature and purified via flash column chromatography (silica, 40% CH₂Cl₂: 60% n-hexane) to afford Compound 16 as a yellow solid (84 mg, 36%).

The name for Compound 20 is 8-(but-3-en-1-yl)-2,3,6,11,12-pentakis(pentyloxy)triphenyleno[1,2-d]oxazole.

Compound 20 had the following characterisation data: ¹H NMR δH: (300 MHz, CDCl₃) 10.00 (1H, s), 7.92 (1H, s), 7.90 (1H, s), 7.89 (1H, s), 7.84 (1H, s), 6.14-6.00 (1H, ddt, J 16.95, 10.20, 6.45 Hz), 5.22 (1H, dd, J 16.95, 1.60 Hz), 5.10 (1H, dd, J 10.20, 1.60 Hz), 4.43-4.35 (4H, m), 4.30-4.24 (6H, m), 3.22-3.17 (2H, t, J 7.55 Hz), 3.17-3.12 (2H, t, J 7.11 Hz), 2.86-2.78 (2H, m), 2.08-1.94 (10H, m), 1.62-1.45 (20H, m), 1.03-0.97 (15H, m) ppm. ¹³C NMR δ_{c}: (100 MHz, CDCl₃) 165.0, 149.8, 149.4, 149.1, 148.7, 143.2, 140.4, 140.0, 137.0, 127.2, 125.0, 124.2, 123.8, 123.6, 116.7, 116.3, 111.4, 108.7, 107.3, 107.2, 103.0, 70.3, 70.2, 69.9, 69.8, 69.2, 30.9, 29.6, 29.5, 29.4, 29.3, 28.9, 28.8, 28.7, 28.6, 28.5, 23.0, 22.9, 14.6, 14.5, 14.5 ppm. ES+m/z: 754.5 ([M+H]⁺ 100%).

Method of Synthesisinq Compound 21

Compound 21 was synthesised using the following method. A solution of decanoic acid (0.132 mg, 0.236 mmol), palladium diacetate (0.005 mmol) and iodobenzene didecanoate (0.235 mmol) in PhMe (10 mL) was heated at 70° C. under N₂ for 20 min. A solution of Precursor 2 (100 mg; 0.132 mmol) in PhMe (10 mL) was added and heated under reflux for 48-72 h, whilst stirring. The solution was cooled to room temperature and diluted with CH₂Cl₂ (20 mL). The organic phase was washed with aqueous NaOH (1M; 2×20 mL), separated and the organic phase was dried in vacuo. The crude black solid was purified by flash column chromatography (silica; 40% CH₂Cl₂: 60% n-hexane) to afford Compound 21 as a white solid (42 mg, 39%).

The name for Compound 21 is 8-nonyl-2,3,6,11,12-pentakis(pentyloxy)triphenyleno[1,2-d]oxazole.

Compound 21 had the following characterisation data: ¹H NMR δH: (300 MHz, CDCl₃) 10.02 (1H, s), 7.93 (1H, s), 7.90 (1H, s), 7.89 (1H, s), 7.84 (1H, s), 4.43-4.36 (4H, m), 4.30-4.24 (6H, m), 3.11-3.06 (2H, t, J 7.52 Hz), 2.10-1.94 (12H, m), 1.63-1.29 (32H, m), 1.02-0.97 (15H, m) 0.91-0.87 (3H, m) ppm. ¹³C NMR δ_{c}: (100 MHz, CDCl₃) 165.9, 149.8, 149.3, 149.0, 148.6, 143.2, 140.4, 140.1, 127.1, 124.9, 124.2, 123.8, 123.6, 123.6, 116.6, 111.3, 108.6, 107.2, 107.1, 102.9, 70.2, 69.9, 69.8, 69.1, 32.3, 29.9, 29.8, 29.7, 29.6, 29.5, 29.4, 29.0 28.9, 28.8, 28.6, 27.1, 23.1, 23.0, 14.6, 14.5 ppm. ES+m/z: 826.6 ([M+H]⁺ 100%).

Method of Synthesisinq Compound 22 Compound 22 was synthesised using the following method. A solution of Precursor 2 (100 mg, 0.132 mmol), 4-(dimethylamino)benzoyl chloride (175 mg, 0.658 mmol) and N,N-Diisopropylethylamine (0.1 mL, 0.574 mmol) in PhMe (5 mL) was heated to and held at reflux for 18 h under N₂. The reaction was cooled to room temperature and then evaporated to dryness in vacuo. The solid was then heated and held at 240° C. for 15 mins under N₂. The crude black solid was then cooled to room temperature and purified via flash column chromatography (silica, 40% CH₂Cl₂: 60% n-hexane) to afford Compound 22 as a yellow solid (19 mg, 18%).

The name for Compound 22 is N,N-dimethyl-4-(2,3,6,11,12-pentakis(pentyloxy)triphenyleno[1,2-d]oxazol-8-yl)aniline.

Compound 22 had the following characterisation data: ¹H NMR δH: (300 MHz, CDCl₃) 10.20 (1H, s), 8.27-8.24 (2H, d, J 8.60 Hz), 7.94 (1H, s), 7.90-7.85 (3H, m), 6.89-6.86 (2H, d, J 8.60 Hz), 4.56-4.45 (4H, m), 4.31-4.24 (6H, m), 3.11 (6H, s), 2.16-1.94 (10H, m), 1.71-1.42 (20H, m), 1.04-0.97 (15H, m) ppm. ¹³C NMR δ_{c}: (100 MHz, CDCl₃) 162.8, 152.2, 149.7, 149.3, 148.5, 143.1, 141.4, 140.2, 129.3, 127.4, 124.9, 124.5, 124.0, 123.6, 116.5, 112.4, 111.5, 108.6, 107.3, 107.2, 103.4, 70.2, 70.2, 69.9, 69.2, 40.9, 29.6, 29.6, 29.5, 29.4, 29.0, 28.9, 28.8, 28.7, 23.0, 23.0, 14.7, 14.5 ppm. ES+m/z: 819.7 ([M]⁺ 100%).

Method of Synthesisinq Compound 23

Compound 23 was synthesised using the following method. A solution of 4-nitrobenzoic acid (1 g, 6 mmol), palladium diacetate (0.005 mmol) and (diacetoxyiodo)benzene (51 mg, 0.157 mmol) in PhMe (10 mL) was heated at 70° C. under N₂ for 20 min. A solution of Precursor 2 (100 mg; 0.132 mmol) in PhMe (10 mL) was added and heated under reflux for 48-72 h, whilst stirring. The solution was cooled to room temperature and diluted with CH₂Cl₂ (20 mL). The organic phase was washed with aqueous NaOH (1M; 2×20 mL), separated and the organic phase was dried in vacuo. The crude black solid was purified by flash column chromatography (silica; 40% CH₂Cl₂: 60% n-hexane) to afford Compound 23 as an off-white solid (74 mg, 69%).

The name for Compound 23 is 8-(4-nitrophenyl)-2,3,6,11,12-pentakis(pentyloxy)triphenyleno[1,2-d]oxazole.

Compound 23 had the following characterisation data: ¹H NMR δ_{H}: (300 MHz, CDCl₃) 9.66 (1H, s), 8.14-8.05 (4H, m), 7.74 (1H, s), 7.73 (1H, s), 7.70 (1H, s), 7.67 (1H, s), 4.30-4.19 (10H, m), 2.04-1.94 (10H, m), 1.63-1.47 (20H, m), 1.05-1.00 (15H, m) ppm. ¹³C NMR δ_{c}: (100 MHz, CDCl₃) 158.9, 150.0, 149.3, 149.1, 148.8, 143.0, 140.6, 140.4, 132.8, 127.8, 127.5, 125.0, 124.0, 123.6, 123.5, 123.3, 116.5, 110.8, 108.3, 106.8, 106.4, 104.3, 70.2, 69.9, 69.8, 69.7, 69.1, 29.7, 29.6, 29.5, 29.4, 28.9, 28.8, 28.8, 28.7, 23.0, 14.6, 14.5 ppm. MALDI m/z: 820.5 ([M]⁺ 100%).

Method of Synthesisinq Compound 24

Compound 24 was synthesised using the following method. A solution of (diacetoxyiodo)benzene (51 mg, 0.157 mmol) and acetylsalicylic acid (550 mg, 3.031 mmol) in toluene (4 mL) was heated to 80° C. and stirred for 10 min under N₂. Then Precursor 2 (100 mg, 0.131 mmol) was added to form a black solution which was then stirred for a further 10 mins. A solution of palladium diacetate (1 mg, 5 mol %) and acetylsalicyclic acid (553 mg, 3.197 mmol) in toluene (4 mL) was heated to 110° C. and stirred for 10 mins under N₂ before being combined with the black solution. The resultant solution was left stirring at 110° C. for 72 h under N₂. The crude black solution was dried in vaccuo and purified by flash column chromatography (silica; 40% CH₂Cl₂: 60% n-hexane). The crude material was then evaporated to dryness in vacuo and then dissolved in a mixture of MeCN (10 mL) and 1M NaOH (10 mL). The solution was heated to 80° C. for 2 h. After cooling to room temperature the product was acidified using 1M HCl (20 mL) and extracted into CH₂Cl₂ (3×10 mL). The combined organic layer was evaporated to dryness in vacuo to afford Compound 24 as a white solid (2 mg. 2%).

The name for Compound 24 is 2-(2,3,6,11,12-pentakis(pentyloxy)triphenyleno[1,2-d]oxazol-8-yl)phenol.

Compound 24 had the following characterisation data: ¹H NMR δ_{H}: (300 MHz, CDCl₃) 11.42 (1H, s), 9.35 (1H, s), 8.20-8.17 (1H, dd, J 8.01, 1.67 Hz), 7.90 (1H, s), 7.89 (1H, s), 7.88 (1H, s), 7.87 (1H, s), 7.52-7.46 (1H, ddd, J 8.51, 7.22, 1.67 Hz), 7.18-7.14 (1H, dd, J 8.51, 1.08 Hz), 7.08-7.05 (1H, ddd, J 8.01, 7.22, 1.08 Hz), 4.47-4.42 (2H, t, J 6.65 Hz), 4.39-4.35 (2H, t, J 6.71 Hz), 4.30-4.19 (6H, m), 2.09-1.94 (10H, m), 1.71-1.43 (20H, m), 1.05-0.98 (15H, m) ppm. MALDI m/z: 791 ([M]⁺ 100%).

Method of Synthesisinq Compound 25

Compound 25 was synthesised using the following method from Precursor 3.

Precursor 3 was fabricated according to the method described in N. Boden et. al. J. Mater. Chem., 1995, 5, 2275. A solution of 2,3,6,7,10,11-hexabutoxy-1-nitrotriphenylene (1.70 g, 2.79 mmol), sodium borohydride (1.70 g, 45.1. mmol) and nickel(II) chloride hexahydrate (4.45 g, 18.7 mmol) in a 50/50 mix of MeOH and THF (40 mL) was stirred at room temperature for 5 h under N₂. The crude black solid was then filtered and washed with CHCl₃ and the filtrate evaporated to dryness in vacuo to afford Precursor 3 as a brown solid (1.6 g, 85%).

The name for Precursor 3 is 2,3,6,7,10,11-hexabutoxytriphenylen-1-amine.

Precursor 3 had the following characterization data: ¹H NMR δ_{H}: (300 MHz, CDCl₃) 8.82 (1H, s), 7.83 (1H, s), 7.80 (1H, s), 7.78 (1H, s), 7.37 (1H, s), 4.57 (2H, s), 4.29-4.09 (12H, m), 2.01-1.81 (12H, m), 1.67-1.52 (12H, m), 1.13-0.94 (18H, m) ppm. ¹³C NMR δ_{c}: (100 MHz, CDCl₃) 151.0, 149.5, 148.9, 148.0, 147.6, 138.4, 135.6, 127.0, 124.7, 124.5, 124.1, 124.0, 114.0, 110.3, 108.5, 108.2, 107.1, 97.4, 72.9, 69.7, 69.4, 69.1, 68.4, 32.7, 31.8, 31.7, 31.6, 31.5, 19.7, 19.6, 19.5, 19.4, 14.1 ppm. MALDI m/z: 675.8 ([M]⁺ 100%).

A solution of Precursor 3 (100 mg, 0.148 mmol), 2-napthoyl chloride (141 mg, 0.658 mmol) and N,N-diisopropylethylamine (0.1 mL, 0.574 mmol) in PhMe (5 mL) was heated to and held at reflux for 18 h under N₂. The reaction was cooled to room temperature and then evaporated to dryness in vacuo.

The solid was then heated and held at 240° C. for 15 mins under N₂. The crude black solid was then cooled to room temperature and purified via flash column chromatography (silica, 40% CH₂Cl₂: 60% n-hexane) to afford Compound 25 as a yellow solid (21 mg, 19%).

The name for Compound 25 is 2,3,6,11,12-pentabutoxy-8-(naphthalen-2-yl)triphenyleno[1,2-d]oxazole.

Compound 25 had the following characterisation data: ¹H NMR δ_{H}: (300 MHz, CDCl₃) 10.01 (1H, s), 8.68 (1H, s), 8.31-8.27 (1H, dd, J 8.57, 1.65 Hz), 7.96-7.87 (3H, m), 7.80-7.79 (3H, m), 7.70 (1H, s), 7.58-7.55 (2H, m), 4.45-4.40 (2H, t, J 7.01 Hz), 4.38-4.34 (2H, t, J 6.67 Hz), 4.28-4.19 (6H, m), 2.15-1.91 (10H, m), 1.79-1.59 (10H, m), 1.18-1.07 (15H, m) ppm. ¹³C NMR δ_{c}: (100 MHz, CDCl₃) 161.6, 149.7, 149.3, 149.0, 148.5, 143.1, 140.8, 140.4, 134.9, 133.4, 129.2, 128.3, 127.8, 127.3, 127.1, 125.0, 124.9, 124.4, 124.1, 123.7, 123.6, 116.6, 111.3, 108.4, 107.1, 106.9, 103.7, 69.8, 69.6, 69.5, 68.9, 31.9, 31.8, 19.9, 19.8, 19.7, 14.5, 14.4, 14.3 ppm. MALDI m/z: 755.1 ([M]⁺ 100%).

Method of Synthesisinq Compound 26

Compound 26 was synthesised using the following method from Precursor 4 (2,3,6,7,10,11-hexakis(pentyloxy)-1,8-triphenylenediamine).

Precursor 4 is synthesised using the method described in N. Boden et. al. J. Mater. Chem., 1995, 5, 2275 from the di-nitro triphenylene derivative, which is formed as a side product in the method that was used to synthesis the mono-nitro triphenylene derivative. The di-nitro triphenylene derivative may be isolated using flash column chromatography in an earlier fraction than the mono-nitro intermediate.

A solution of Precursor 4 (135 mg, 0.174 mmol) and palladium diacetate (0.0005 mmol) in PhMe (7 mL) was heated at reflux under N₂ for 10 min. 2-flourobenzoyl chloride (0.02 mL, 0.174 mmol) was added and heated under reflux for 40 h. The solution was cooled to room temperature and dried in vacuo and the resultant black solid was heated at 240° C. for 10 minutes before being cooled to room temperature. The crude black solid was purified by flash column chromatography (silica; 40% CH₂Cl₂: 60% n-hexane) to afford Compound 26 as an off-white solid (6 mg, 4%).

The name for Compound 26 is 2,9-bis(2-fluorophenyl)-4,7,12,13-tetrakis(pentyloxy)triphenyleno[1,2-d:8,7-d′]bis(oxazole).

Compound 26 had the following characterisation data: ¹H NMR δ_{H}: (300 MHz, CDCl₃) 10.09 (2H, s), 8.38-8.32 (2H, td, J 7.56, 7.52, 1.78 Hz), 7.82 (2H, m), 7.58 (2H, m), 7.36-7.31 (2H, m), 7.29-7.25 (2H, m), 4.48-4.39 (8H, m), 2.16-1.96 (8H, m), 1.70-1.44 (16H, m), 1.05-1.00 (12H, m) ppm. ¹⁹F NMR δ_{F}: (300 MHz, CDCl₃) 108.9 (s) ppm. MALDI m/z: 842.5 ([M]⁺ 100%).

Method of Synthesisinq Compound 27

Compound 27 was synthesised using the following method. A solution of 4′-Carboxybenzo-15-crown-5 (600 mg, 1.92 mmol), Oxalyl chloride (2.0 mL, 23.6 mmol) and Dimethylformamide (0.01 ml, 0.129 mmol) was heated and held at reflux for 10 minutes under N₂. The reaction was cooled to room temperature and then evaporated to dryness in vacuo. A solution of Precursor 2 (100 mg, 0.132 mmol) and N,N-Diisopropylethylamine (0.1 mL, 0.574 mmol) in PhMe (5 mL) was added. The reaction was then heated and held at reflux for 72 h under N₂. The reaction was cooled to room temperature and then evaporated to dryness in vacuo. The solid was then heated and held at 240° C. for 15 mins under N₂. The crude black solid was then cooled to room temperature and purified via flash column chromatography (silica, 10% EtOAc: 90% n-hexane) to afford compound 27 as a brown solid (22 mg, 17%).

The name for Compound 27 is 8-(2,3,5,6,8,9,11,12-octahydrobenzo[b][1,4,7,10,13]pentaoxacyclopentadecin-15-yl)-2,3,6,11,12-pentakis(pentyloxy)triphenyleno[1,2-d]oxazole.

Compound 27 had the following characterisation data: ¹H NMR δ_{H}: (300 MHz, CDCl₃) 10.10 (1H, s), 8.04-7.80 (6H, m), 6.99 (1H, d, J 8.35 Hz), 4.53-4.38 (4H, m), 4.33-4.17 (10H, m), 4.06-3.90 (4H, m), 3.89-3.73 (8H, m), 2.10-1.88 (10H, m), 1.69-1.44 (20H, m), 1.07 0.92 (15H, m) ppm. ¹³C NMR δ_{c}: (100 MHz, CDCl₃) 161.6, 152.1, 149.6, 149.1, 148.9, 148.4, 142.9, 140.7, 140.1, 127.2, 124.7, 124.0, 123.6, 123.4, 121.7, 120.5, 116.4, 113.2, 112.9, 111.1, 108.3, 107.0, 103.4, 71.3, 70.6, 70.5, 70.0, 70.0, 69.9, 69.6, 69.5, 69.2, 68.9, 68.7, 29.8, 29.4, 29.3, 29.2, 28.7, 28.5, 28.4, 22.8, 22.7, 14.4, 14.3 ppm. MALDI m/z: 965.9 ([M]⁺ 100%).

Method of Synthesisinq Compound 28

Compound 28 was synthesised using the following method. A solution of Compound 2 (60 mg, 0.077 mmol) in degassed dichloromethane (5 mL) was stirred in a nitrogen purged 2 neck flask under nitrogen atmosphere at −20° C. Boron tribromide (1M solution in CH₂Cl₂, 387 μL, 0.385 mmol, 5 eq) was added via syringe through a Suba-Seal(RTM) and the dark yellow solution was stirred for at room temperature for 24 h. Water (40 mL) was added to quench the reaction and the product was extracted with dichloromethane (10 mL), washed with water (2×20 mL) and dried over MgSO₄. The organic phase was evaporated to dryness and purified by column chromatography (Silica: 5% Ethyl acetate: Hexane) to afford Compound 28 as a brown solid (10 mg, 6%).

The name for Compound 28 is 2,3,11,12-tetrakis(pentyloxy)-8-phenyltriphenyleno[1,2-d]oxazol-6-ol.

Compound 28 had the following characterisation data: ¹H NMR (300 MHz, CDCl₃) δ_{H}: 10.09 (1H, s), 8.48 (2H, dd, J 6.86, 2.85 Hz), 7.94 (1H, s), 7.90 (1H, s), 7.88 (1H, s), 7.83 (1H, s), 7.59-7.56 (3H, m), 5.95 (1H, s br), 4.48 (2H, t, J 6.60 Hz), 4.33 (2H, t, J 6.51 Hz), 4.26-4.24 (4H, m), 2.06-1.93 (8H, m), 1.64-1.43 (16H, m), 1.01-0.97 (12H, m) ppm. ES⁺MS m/z: 728.4 ([M+Na]⁺ 25%), 707.4 ([M+H+1]⁺ 30%), 706.4 ([M+H]⁺ 85%).

Method of Synthesisinq Compound 29

Compound 29 was synthesised using the following method. Compound 2 (250 mg, 0.322 mmol) was dissolved in dry CH₂Cl₂ (10 mL) and stirred at 0° C. under a N₂ atmosphere. 0.01M solution of Br₂ in CH₂Cl₂ (144 mL, 1.449 mmol) was then added over 2 h (4×36 mL) and monitered by TLC. The reaction was quenched by addition of saturated sodium metabisulfate solution (100 mL). The product was extracted with dichloromethane (30 mL) washed with water (3×30 mL), dried over MgSO₄ and evaporated to dryness. The crude product was then purified by column chromatography (Silica 40% CH₂Cl₂: n-hexane) to yield Compound 29 as a yellow solid (170 mg, 62%).

The name for Compound 29 is 1-bromo-2,3,6,11,12-pentakis(pentyloxy)-8-phenyltriphenyleno[1,2-d]oxazole.

Compound 29 had the following characterisation data: ¹H NMR (300 MHz, CDCl₃) δ_{H}: 10.06 (1H, s), 8.71 (1H, s), 8.56 (1H, s), 8.34-8.31 (2H, m), 7.60-7.58 (4H, m), 4.59 (2H, t, J 6.5 Hz), 4.41 (2H, t, J 6.8 Hz), 4.26-4.20 (6H, m), 2.06-1.90 (10H, m), 1.64-1.42 (20H, m), 1.02-0.94 (15H, m) ppm.

Method of Synthesisinq Compound 30

Compound 30 was synthesised using the following method. Compound 29 (170 mg, 0.199 mmol), K₂CO₃ (410 mg, 2.97 mmol) and Pd(PPh₃)₄ (30 mg, 0.026 mmol) were dissolved in degassed 5: 1 THF:H₂O mix (4 mL) under nitrogen atmosphere. (4-hydroxyphenyl)boronic acid (140 mg, 1.02 mmol) was then added and the reaction was heated to reflux under N₂ for 24 h. The product was extracted with dichloromethane (30 mL), washed with water (3×30 mL) and evaporated to dryness. The crude product was purified by column cromatography (Silica CH₂Cl₂: n-hexane) to yield impure Compound 30 as a brown solid.

The name for Compound 30 is 4-(2,3,6,11,12-pentakis(pentyloxy)-8-phenyltriphenyleno[1,2-d]oxazol-1-yl)phenol.

Compound 30 had the following characterisation data: TOF LD*⁺ m/z: 869.49 ([M+1]⁺ 70%), 868.52 ([M]⁺ 70%).

Method of Synthesisinq Compound 31

Compound 31 was synthesised using the following method. Compound 10 (50.7 mg, 0.071 mmol) was added to a 2 neck round bottom flask, which was purged with N₂ for 15 min. Dry CH₂Cl₂ (20 mL) was then added via syringe through a Suba-Seal(RTM) and the brown stirring solution was cooled to −78° C. Boron tribromide (1M solution in CH₂Cl₂, 391 μL, 0.391 mmol, 5.5 eq) was added via syringe through a Suba-Seal(RTM) and the reaction was stirred for 4 h. The reaction mixture was poured over crushed ice and stirred until the ice had fully melted, 4 drops of hydrochloric acid (1M) were added and the product was extracted with ethyl acetate, washed with water (2×20 mL), and dried over MgSO₄ and evaporated to dryness. The crude product was used without further purification.

The name for Compound 31 is 8-methyltriphenyleno[1,2-d]oxazole-2,3,6,11,12-pentaol.

Compound 31 had the following characterisation data: ES⁺*MS m/z: 503.26 ([M+2(OC₅H₁₁)]⁺ 50%), 433.17 ([M+(OC₅H₁₁)]⁺ 100%), 363.08 ([M]⁺ 10%).

Method of Synthesisinq Compound 32

Crude Compound 31 (26 mg, 0.071 mmol), potassium carbonate (74 mg, 0.533 mmol), potassium iodide (6 mg, 0.036 mmol) was dissolved in dry MeCN (35 mL). 1(-2-Bromoethoxy)-2-(2-methoxyethoxy)ethane (132 μL, 0.533 mmol) was then added via pipette and the reaction was heated to reflux and stirred under a CaCl₂ drying tube for 20 h. The reaction was cooled to room temperature and the product was extracted with ethyl acetate (20 mL), washed with water (3×20 mL), brine (2×20 mL) and dried over MgSO₄ to yield crude Compound 32 as a brown solid.

The name for Compound 32 is 2,3,6,12-tetrakis(2-(2-(2-methoxyethoxy)ethoxy)ethoxy)-8-methyl-11-(pentyloxy)triphenyleno[1,2-d]oxazole.

Compound 32 had the following characterisation data: ES⁺MS m/z: 1017.5 ([M]⁺ 100%).

Advantageously, Compound 32 is water soluble.

Method of Synthesisinq Compound 33

Compound 33 was synthesised from Precursor 5 in the following method. The starting material to form Precursor 5 was obtained using the method described in J. Mater. Chem. C, 2017, 5, 669-682 (DOI: 10.1039/C6TC04530H).

5,6-Dimethoxy-2,3,8,9,12,13-hexakis(pentyloxy)dibenzo[fg,op]tetracene (681 mg, 0.775 mmol) was dissolved in diethyl ether (20 ml) and then acetic acid (1.33 ml, 23.24 mmol, 30 eqiv) was added and the mixture stirred at room temperature in a nitrogen atmosphere for 10 minutes before the addition of fuming nitric acid (65 μL, 1.55 mmol, 2 equiv) was added. The reaction mixture was stirred under nitrogen for 20 min before the further addition of nitric acid (30 μL, 0.715 mmol, 0.92 eq) and the reaction mixture was left for 20 min stirring at room temperature. The mixture was then quenched with water (10 ml) and the organic phase was washed with NaOH (1M, 2×30 mL) and then dried in vacuo to provide Precursor 5 as a black solid (680 mg, 95%). This was used in the next step with no further purification.

The name for Precursor 5 is 5,6-dimethoxy-1-nitro-2,3,8,9,12,13-hexakis(pentyloxy)dibenzo[fg,op]tetracene.

Precursor 5 had the following characterisation data: ¹H NMR (400 MHz, CDCl₃) δ_{H} 9.16 (1H, s), 9.01 (1H, s), 7.95 (1H, s), 7.84 (1H, s), 7.58 (1H, s), 4.53 (2H, t, J 6.7 Hz), 4.32 (2H, t, J 5.1 Hz), 4.29 (2H, t, J 5.3 Hz), 4.18-4.14 (2H, m), 4.14 (3H, s), 4.13 (3H, s), 3.95 (2H, t, J 6.8 Hz), 3.95 (2H, t, J 7.0 Hz), 2.11-1.82 (12H, m), 1.69-1.29 (24H, m), 1.08-0.84 (18H, m). ¹³C NMR (101 MHz, CDCl₃) δ_{c} 151.9, 149.6, 149.3, 148.7, 148.1, 147.9, 144.4, 143.2, 141.5, 124.9, 124.8, 124.7, 124.4, 123.1, 123.1, 122.3, 119.4, 118.6, 116.0, 109.5, 109.3, 107.6, 107.3, 104.9, 76.3, 74.5, 74.0, 69.5, 68.9, 68.8, 55.8, 30.3, 30.3, 29.9, 29.2, 29.0, 28.8, 28.4, 28.3, 28.2, 28.1, 22.6, 22.6, 22.6, 14.1, 14.0, 14.0. MALDI₊ m/z: 924.82 ([M+H]⁺ 30%).

Precursor 6 was synthesised in the following method. Precursor 5 (680 mg, 0.736 mmol) and NiCl₂.6H₂O (552 mg, 2.33 mmol, 3 equivalents) were dissolved in THF:MeOH (20 mL, 5:4 ratio), to make a yellow solution, and then NaBH₄ (586 mg, 15.5 mmol, 20 equivalents) was added over 15 minutes. The black reaction mixture was left stirring under a N₂ atmosphere for 40 min after which time it was diluted with chloroform and the precipitate was gravity filters to leave a brown organic phase, which was then dried in vacuo to provide Precursor 6 as brown solid (614 mg, 93%).

The name for Precursor 6 is 5,6-dimethoxy-2,3,8,9,12,13-hexakis(pentyloxy)dibenzo[fg,op]tetracen-1-amine.

Precursor 6 had the following characterisation data: ¹H NMR (300 MHz, CDCl₃) δ_{H} 9.09 (1H, s), 8.98 (1H, s), 8.67 (1H, s), 7.97 (1H, s), 7.90 (1H, s), 4.76 (2H, s), 4.40 (2H, t, J 6.8 Hz), 4.34 (2H, t, J 6.5 Hz), 4.28 (2H, t, J 6.6 Hz), 4.20 (2H, t, J 6.6 Hz), 4.11 (3H, s), 4.08 (3H, s), 3.99 (2H, t, J 7.0 Hz), 3.93 (2H, t, J 7.0 Hz), 2.07-1.88 (12H, m), 1.66-1.34 (24H, m), 1.06-0.88 (18H, m, J 22.0, 12.0, 7.1 Hz). ¹³C NMR (101 MHz, CDCl₃) δ_{c} 151.2, 148.5, 148.2, 148.0, 146.8, 143.8, 139.8, 136.9, 125.4, 125.0, 124.5, 124.4, 123.6, 123.5, 122.6, 120.5, 113.9, 111.8, 110.0, 109.4, 108.6, 108.4, 104.3, 73.9, 73.8, 69.7, 69.2, 69.0, 55.7, 30.4, 30.3, 29.7, 29.3, 29.1, 29.0, 28.5, 28.4, 28.3, 22.7, 22.6, 22.6, 14.1, 14.0. MALDI⁺ m/z: 893.8 ([M+H]⁺ 100%).

Precursor 7 was synthesised in the following method. Precursor 6 (147 mg, 0.181 mmol) was dissolved in dry CH₂Cl₂ (20 mL) and dry MeCN (20 mL). The solution was cooled to 0° C. under a N₂ atmosphere then tert-butyl nitrite (34 μL, 0.309 mmol, 1.7 equivalents) and TMSN₃ (36 μL, 0.273 mmol, 1.5 equivalents) were added and the reaction mixture stirred 0° C. for 10 min and then at room temperature for 20 min. The solution was then dried in vacuo and purified via flash column chromatography (silica, 30% DCM, 70% n-hexane) to provide Precursor 7 as a while solid (120 mg, 72%).

The name for Precursor 7 is 1-azido-5,6-dimethoxy-2,3,8,9,12,13-hexakis(pentyloxy)dibenzo[fg,op]tetracene.

Precursor 7 had the following characterisation data: ¹H NMR (300 MHz, CDCl₃) δ_{H} 9.12 (1H, s), 9.03 (1H, s), 8.98 (1H, s), 8.01 (1H, s), 7.89 (1H, s), 4.45 (2H, t, J 6.7 Hz), 4.38-4.32 (2H, m), 4.29 (2H, t, J=6.0 Hz), 4.11 (3H, s), 4.09 (3H, s), 4.01 (2H, m), 3.94 (2H, t, J 6.6 Hz), 2.13-1.84 (12H, m), 1.69-1.32 (24H, m), 1.04-0.87 (18H, m). ¹³C NMR (101 MHz, CDCl₃) δ_{c} 151.6, 148.9, 148.0, 148.0, 147.9, 147.9, 146.3, 144.1, 127.3, 125.4, 124.8, 124.0, 123.9, 123.3, 123.2, 122.8, 120.2, 119.4, 117.6, 112.6, 109.4, 109.1, 107.5, 104.7, 75.3, 74.4, 74.0, 69.6, 69.1, 69.0, 55.8, 30.4, 30.3, 29.5, 29.3, 29.1, 28.9, 28.5, 28.3, 28.2, 28.1, 22.7, 22.6, 22.5, 14.1, 14.0.

Compound 33 was synthesised using the following method. Precursor 7 (100 mg; 0.13 mmol) was dissolved in dry PhMe (5 mL) was added to a flask containing rhodium octanoate dimer (5 mg; 0.01 mmol), under a N₂ atmosphere. This mixture was then heated to reflux and stirred for 20 hours. The reaction was cooled to room temperature and then dried in vacuo, the solid was then purified via flash column chromatography (silica; 95% n-hexane: 5% ethyl acetate) to provide Compound 33 as a white solid (58 mg, 50%).

The name of Compound 33 is 2-butyl-12,13-dimethoxy-5,6,9,10,15-pentakis(pentyloxy)dibenzo[4,5:9,10]pyreno[1,2-d]oxazole.

Compound 33 had the following characterisation data: ¹H NMR (300 MHz, CDCl₃) δ_{H} 10.08 (1H, s), 9.28 (1H, s), 9.15 (1H, s), 8.16 (1H, s), 8.02 (1H, s), 4.42 (2H, t, J 6.0 Hz), 4.37 (2H, d, J=5.8 Hz), 4.35-4.29 (4H, m, J 6.7, 3.3 Hz), 4.12 (3H, s), 4.11 (3H, s), 3.98 (2H, t, J=6.9 Hz), 3.18 (2H, t, J=7.5 Hz), 2.09-1.92 (10H, m), 1.67-1.36 (24H, m), 1.07-0.90 (18H, m). ¹³C NMR (101 MHz, CDCl₃) δ_{c} 166.1, 151.3, 149.5, 148.8, 147.8, 147.7, 144.0, 142.4, 138.4, 137.1, 125.5, 124.9, 124.1, 124.0, 123.7, 123.5, 123.0, 119.9, 118.8, 116.1, 111.3, 109.6, 109.4, 107.1, 104.8, 74.5, 74.0, 69.8, 69.0, 68.8, 55.8, 55.7, 30.3, 30.1, 29.3, 29.1, 28.9, 28.7, 28.5, 28.4, 28.3, 28.2, 22.6, 22.5, 22.3, 14.1, 14.0, 13.8. MALDI⁺ m/z: 889.2 ([M+H]⁺ 100%).

Method of Synthesisinq Compound 34

Precursor 6 (47 mg; 0.053 mmol), benzoyl chloride (30 μL, 0.265 mmol, 5 equivalents), and diisopropylethylamine (46 μL, 0.265 mmol, 5 equiv) were dissolved in dry PhMe (5 mL) and the mixture was heated to reflux under a N₂ atmosphere. The reaction mixture was stirred for 1 h at which point the mixture was dried in vacuo then the solid was heated to 240° C. for 10 min. The reaction was cooled to room temperature and the solid was then purified via flash column chromatography (silica; 40% CH₂Cl₂, 60% n-hexane) to afford Compound 34 a white solid (16 mg, 33%).

The name of Compound 34 is12,13-dimethoxy-5,6,9,10,15-pentakis(pentyloxy)-2-phenyldibenzo[4,5:9,10]pyreno[1,2-d]oxazole.

Compound 34 had the following characterisation data: ¹H NMR (300 MHz, CDCl₃) δ_{H} 9.29 (1H, s), 9.19 (1H, s), 8.48-8.43 (2H, m), 8.17 (1H, s), 8.03 (1H, s), 7.65-7.59 (3H, m), 4.53 (2H, t, J=6.9 Hz), 4.44-4.33 (6H, m), 4.14 (3H, s), 4.12 (3H, s), 3.99 (2H, t, J=6.9 Hz), 2.15-1.90 (10H, m), 1.71-1.36 (24H, m), 1.06-0.80 (18H, m). ¹³C NMR (101 MHz, CDCl₃) δ_{c} 166.6, 161.8, 151.4, 149.5, 148.9, 147.9, 147.9, 144.1, 142.5, 138.5, 137.7, 132.9, 131.5, 129.5, 129.0, 128.4, 127.6, 127.4, 125.6, 124.8, 124.3, 124.0, 123.8, 123.5, 123.3, 119.8, 119.7, 116.4, 111.3, 109.7, 109.4, 106.9, 104.8, 74.7, 74.1, 69.8, 69.0, 64.5, 55.8, 55.7, 30.4, 30.2, 29.7, 29.3, 29.1, 28.9, 28.5, 28.5, 28.4, 25.6, 22.6, 22.5, 14.2, 14.1, 14.1, 14.1, 14.0. ES⁺ m/z: 910.5 ([M+H]⁺ 100%).

Method of Synthesisinq Compound 35

Precursor 6 (100 mg; 0.111 mmol) 4-cyanobenzoyl chloride (92 mg, 0.555 mmol, 5 equiv), and diisopropylethylamine (90 μL, 0.555 mmol, 5 equiv) were dissolved in dry toluene (5 mL) and the mixture was heated reflux under a nitrogen atmosphere. The reaction mixture was stirred for 1 hour at which point the mixture was dried in vaccuo then the solid was heated to 240° C. for 10 minutes. The reaction was cooled to room temperature and the solid was then purified via flash column chromatography (silica; 40% DCM, 60% n-hexane) to afford Compound 35 as a white solid (17 mg, 16%).

The name of Compound 35 is 4-(12,13-dimethoxy-5,6,9,10,15-pentakis(pentyloxy)dibenzo[4,5:9,10]pyreno[1,2-d]oxazol-2-yl)benzonitrile.

Compound 35 had the following characterisation data: ¹H NMR (300 MHz, CDCl₃) δ 10.02 (s, 1H), 9.27 (s, 1H), 9.15 (s, 1H), 8.43 (d, J=8.3 Hz, 2H), 8.13 (s, 1H), 7.99 (s, 1H), 7.84 (d, J=8.3 Hz, 2H), 4.45 (t, J=6.8 Hz, 2H), 4.36 (dt, J=13.0, 6.4 Hz, 6H), 4.14 (s, 3H), 4.12 (s, 3H), 3.97 (t, J=6.9 Hz, 2H), 2.12-1.90 (m, 10H), 1.65-1.38 (m, 24H), 1.06-0.88 (m, 18H). ¹³C NMR (100 MHz, CDCl₃) δ 159.8, 151.9, 149.9, 149.5, 148.5, 148.3, 144.6, 143.0, 138.6, 137.6, 133.0, 131.5, 128.0, 125.8, 124.8, 124.4, 123.9, 123.8, 123.8, 121.1, 119.9, 118.6, 117.0, 114.7, 111.5, 110.1, 109.8, 107.1, 105.2, 75.1, 74.4, 70.1, 69.3, 56.2, 56.1, 30.7, 30.5, 30.0, 29.6, 29.5, 29.3, 28.9, 28.8, 28.7, 28.7, 23.0, 22.9, 22.9, 14.6, 14.5, 14.4. MALDI⁺ m/z: 934.55 ([M+H]⁺ 100%).

Method of Synthesisinq Compound 36

Compound 1 was synthesised using the following method. A solution of Precursor 1 (100 mg, 0.132 mmol), benzoyl chloride (92 mg, 0.658 mmol) and N,N-diisopropylethylamine (0.1 mL, 0.574 mmol) in PhMe (5 mL) was heated to and held at reflux for 18 h under N₂. The reaction was cooled to room temperature and then evaporated to dryness in vacuo purified via flash column chromatography (silica, 60% CH₂Cl₂: 40% n-hexane) to afford an intermediate as a brown solid (19 mg, 18%).

The intermediate had the following characterisation data: ¹H NMR δ_{H}: (300 MHz, CDCl₃) 8.55 (1H, s), 8.45 (1H, s), 8.07 (2H, d, J 7.5 Hz), 7.78 (1H, s), 7.74 (1H, s), 7.72 (1H, s), 7.71 (1H, s), 7.59 (1H, d, J 7.0 Hz), 7.54 (2H, t, J 7.4 Hz), 4.28-4.12 (10H, m), 3.67-3.54 (2H, m), 2.00-1.85 (8H, m), 1.70-1.37 (20H, m), 1.34-1.06 (8H, m), 1.02-0.90 (12H, m), 0.83 (3H, t, J 7.0), 0.75 (3H, t, J 7.1) ppm. ¹³C NMR δ_{c}: (100 MHz, CDCl₃) 174.6, 151.0, 149.7, 148.7, 148.4, 148.4, 144.0, 135.1, 131.7, 130.9, 128.5, 127.9, 126.6, 124.7, 124.2, 123.0, 122.6, 122.0, 110.3, 108.1, 107.7, 106.8, 106.7, 73.4, 70.1, 70.0, 69.5, 69.3, 68.8, 32.1, 30.1, 29.9, 29.6, 29.4, 29.3, 28.7, 28.5, 28.5, 28.1, 22.9, 22.7, 22.6, 14.3, 14.3, 14.1 ppm. MALDI m/z: 863.3 ([M]+100%).

A solution of the intermediate (100 mg, 0.116 mmol) and Lawesson's Reagent (175 mg, 0.658 mmol) in PhMe (5 mL) was heated to and held at reflux for 48 h under N₂. The reaction was cooled to room temperature and then evaporated to dryness in vacuo. The solid was then heated and held at 240° C. for 15 min under N₂. The crude black solid was then cooled to room temperature and purified via flash column chromatography (silica, 40% CH₂Cl₂: 60% n-hexane) to afford Compound 36 as a green solid (17 mg, 19%).

The name for Compound 36 is 2,3,6,11,12-pentakis(pentyloxy)-8-phenyltriphenyleno[1,2-d]thiazole.

Compound 36 had the following characterisation data: ¹H NMR δ_{H}: (300 MHz, CDCl₃) 10.51 (1H, s), 8.24-8.22 (2H, m), 7.92-7.89 (3H, m), 7.76 (1H, s), 7.53-7.52 (3H, m), 4.43-4.26 (10H, m), 2.10-1.95 (10H, m), 1.66-1.57 (10H, m), 1.53-1.47 (10H, m), 1.03-1.00 (15H, m) ppm. ¹³C NMR δ_{c}: (100 MHz, CDCl₃) 166.4, 152.5, 151.5, 150.2, 149.3, 148.3, 134.7, 130.9, 130.0, 129.3, 127.6, 125.7, 125.4, 124.7, 123.8, 119.0, 112.4, 108.9, 107.2, 106.9, 100.9, 70.3, 70.2, 69.7, 69.2, 69.1, 29.7, 29.6, 29.6, 29.5, 29.4, 28.8, 28.8, 28.8, 23.1, 23.0, 23.0, 23.0 23.0, 14.5, 14.5, 14.5, 14.5 ppm. MALDI m/z: 791.6 ([M]+100%).

Method of Synthesisinq Compound 37

Compound 37 was synthesised using the following method. A solution of Precursor 1 (100 mg, 0.132 mmol), 4-cyanobenzoyl chloride (109 mg, 0.658 mmol) and N,N-diisopropylethylamine (0.1 mL, 0.574 mmol) in PhMe (5 mL) was heated to and held at reflux for 18 h under N₂. The reaction was cooled to room temperature and then evaporated to dryness in vacuo. The crude brown solid was added to a solution of Lawesson's Reagent (175 mg, 0.658 mmol) in PhMe (5 mL) was heated to and held at reflux for 48 h under N₂. The reaction was cooled to room temperature and then evaporated to dryness in vacuo. The solid was then heated and held at 240° C. for 15 min under N₂. The crude black solid was then cooled to room temperature and purified via flash column chromatography (silica, 40% CH₂Cl₂: 60% n-hexane) to afford Compound 37 as a yellow solid (5 mg, 5%).

The name for Compound 37 is 4-(2,3,6,11,12-pentakis(pentyloxy)triphenyleno[1,2-d]thiazol-8-yl)benzonitrile.

Compound 37 had the following characterisation data: ¹H NMR δ_{H}: (300 MHz, CDCl₃) 10.38 (1H, s), 8.31 (2H, d, J 8.4), 7.97-7.88 (4H, m), 7.79 (1H, d, J 8.5), 4.41-4.26 (10H, m), 2.06-1.95 (10H, m), 1.61-1.55 (10H, m), 1.51-1.44 (10H, m), 1.03-0.97 (15H, m) ppm. MALDI m/z: 816.9 ([M]+90%), 817.9 ([M+H]⁺ 100%).

Method of Synthesisinq Compound 38

Compound 38 was synthesised from Precursor 8 in the following method.

Precursor 8 was synthesised using the following method. Compound 3 (8-(naphthalen-2-yl)-2,3,6,11,12-pentakis(pentyloxy)triphenyleno[1,2-d]oxazole) (150 mg, 0.18 mmol, 1 eq) was dissolved in CH₂Cl₂ (10 mL) which had been dried and degassed for 10 min in a 2 necked flask fitted with a Subaseal(RTM) and had been purged with N₂ for 10 min. The green solution was stirred at −10° C. under N₂ for 10 min and boron tribromide (1M in CH₂Cl₂ solution) (0.2 mmol, 1.1 eq) was added via syringe through the Subaseal(RTM). The black solution was stirred under N₂ for 2 h at −10° C. and then poured over crushed ice and stirred until all the ice had melted. The product was then extracted with ethyl acetate (20 mL), washed with water (3×50 mL) and evaporated to dryness yielding a brown solid. The product was then purified by flash column chromatography (20% EtOAc: n-hexane, silica) yielding 8-(naphthalen-2-yl)-2,3,6,12-tetrakis(pentyloxy)triphenyleno[1,2-d]oxazol-11-ol as a yellow solid (31 mg, 23%).

The name for Precursor 8 is 8-(naphthalen-2-yl)-2,3,6,12-tetrakis(pentyloxy)triphenyleno[1,2-d]oxazol-11-ol.

Precursor 8 had the following characterisation data-¹H NMR δ_{H}: (300 MHz, CDCl₃) 10.06 (1H, s), 8.83 (1H, s), 8.50 (1H, dd, J 8.6, 1.7), 8.05-8.03 (1H, m), 7.79 (1H, d, J 8.7), 7.91-7.89 (1H, m), 7.84 (1H, s), 7.79 (1H, s), 7.78 (1H, s), 7.74 (1H, s), 7.58-7.56 (2H, m), 5.97 (1H, s), 4.42 (2H, t, J 6.7), 4.29-4.20 (6H, m), 2.05-1.93 (8H, m), 1.63-1.45 (16H, m), 1.05-0.98 (12H, m) ppm. ¹³C NMR δ_{c}: (100 MHz, CDCl₃) 161.7, 149.4, 148.9, 146.0, 145.3, 143.0, 140.9, 140.2, 134.8, 133.2, 129.1, 128.66, 128.0, 127.9, 127.6, 127.2, 126.8, 124.8, 124.7, 124.7, 124.0, 123.6, 123.4, 116.4, 112.7, 108.1, 107.1, 103.7, 103.6, 69.9, 69.8, 69.7, 69.1, 29.3, 29.3, 29.2, 28.6, 28.5, 28.4, 22.8, 22.7, 14.3, 14.2 ppm. MALDI m/z: 755.65 ([M]⁺ 100%), 756.66 ([M+H]⁺ 70%), 757.67 ([M+1+H]⁺ 25%).

A slurry of Precursor 8 (100 mg, 0.13 mmol) and K₂CO₃ (114 mg, 0.83 mmol) in DMF (5 mL) was heated at reflux with a CaCl₂ drying tube for 0.5 h, followed by addition of methyl 2-(2-(2-chloroethoxy)ethoxy)acetate (114 mg, 0.40 mmol) and KI (75 mg, 0.5 mmol). The resultant slurry was further heated at reflux for 3 h. The reaction mixture was allowed to cool to room temperature and the precipitate filtered off via suction filtration. The solvent was evaporated from the filtrate to dryness in vacuo and the crude solid was purified by purified via flash column chromatography (silica, 25% EtOAc: 75% n-hexane) to afford Compound 38 as a yellow solid (36 mg, 30%).

The name for Compound 38 is methyl 2-(2-(2-((8-(naphthalen-2-yl)-2,3,6,12-tetrakis(pentyloxy)triphenyleno[1,2-d]oxazol-11-yl)oxy)ethoxy)ethoxy)acetate.

Compound 38 had the following characterisation data: ¹H NMR δ_{H}: (300 MHz, CDCl₃) 10.16 (1H, s), 8.83 (1H, s), 8.50 (1H, dd, J 8.6, 1.7), 8.05-8.03 (2H, m), 7.91-7.81 (5H, m), 7.58-7.56 (2H, m), 4.60 (2H, t, J 5.5), 4.42 (2H, t, J 6.7), 4.29-4.21 (6H, m), 4.20 (3H, s), 3.85-3.65 (8H, m), 2.05-1.93 (8H, m), 1.63-1.45 (16H, m), 1.05-0.98 (12H, m) ppm. ¹³C NMR δ_{c}: (100 MHz, CDCl₃) 173.9, 161.7, 149.4, 148.9, 145.9, 145.3, 142.0, 140.8, 140.4, 134.8, 133.1, 129.1, 128.7, 128.0, 127.9, 127.6, 127.2, 126.8, 124.8, 124.7, 124.6, 124.0, 123.6, 123.3, 116.5, 112.7, 108.1, 107.1, 103.7, 103.6, 71.8, 71.4, 71.2, 70.4, 69.8, 69.8, 69.7, 69.0, 68.5, 29.4, 29.3, 29.2, 28.6, 28.5, 28.4, 22.8, 22.7, 14.3, 14.2 ppm. MALDI m/z: 915.7 ([M]⁺ 100%), 916.7 ([M+H]⁺ 90%). Elemental analysis Found: C, 73.41; H, 7.56; N, 1.54%. C₅₆H₆₉NO₁₀ requires C, 73.42; H, 7.59; N, 1.53%.

Method of Synthesisinq Compound 39

Compound 39 was synthesised using the following method. A solution of NaOH (3 mg, 0.08 mmol) in H₂O (1 mL) was added to a solution of Compound 38 (35 mg, 0.04 mmol in MeOH (5 mL). The resultant solution was heated at reflux for 5 h. The reaction mixture was allowed to cool to room temperature and aliquots of 1M HCl aqueous solution was added until no further precipitate was 55 formed. The precipitate was collected through suction filtration affording a yellow solid (24 mg, 70%).

The name for Compound 39 is 2-(2-(2-((8-(naphthalen-2-yl)-2,3,6,12-tetrakis(pentyloxy)triphenyleno[1,2-d]oxazol-11-yl)oxy)ethoxy)ethoxy)acetic acid.

Compound 39 had the following characterisation data: ¹H NMR δ_{H}: (300 MHz, CDCl₃) 10.16 (1H, s), 8.83 (1H, s), 8.50 (1H, dd, J 8.6, 1.6), 8.05-8.03 (2H, m), 7.91-7.81 (5H, m), 7.58-7.56 (2H, m), 4.60 (2H, t, J 5.5 Hz), 4.42 (2H, t, J 6.7 Hz), 4.29-4.21 (6H, m), 3.85-3.65 (8H, m), 2.05-1.93 (8H, m), 1.63-1.45 (16H, m), 1.05-0.98 (12H, m) ppm. ¹³C NMR δ_{c}: (100 MHz, CDCl₃) 175.7, 149.4, 148.9, 145.9, 145.3, 142.0, 140.8, 140.4, 134.8, 133.1, 129.1, 128.7, 128.0, 127.9, 127.6, 127.2, 126.8, 124.8, 124.7, 124.6, 124.0, 123.6, 123.3, 116.5, 112.7, 108.1, 107.1, 103.7, 103.6, 71.8, 71.4, 71.2, 70.4, 69.8, 69.8, 69.7, 69.0, 68.5, 29.4, 29.3, 29.2, 28.6, 28.5, 28.4, 22.8, 22.7, 14.3, 14.2 ppm. MALDI m/z: 901.5 ([M]⁺ 100%), 902.5 ([M+H]⁺ 70%). Elemental analysis Found: C, 73.24; H, 7.52; N, 1.54%. C₅₅H₆₇NO₁₀ requires C, 73.23; H, 7.55; N, 1.55%.

Method of Synthesisinq Compound 40

Compound 40 was synthesised using the following method. A slurry of Precursor 8 (100 mg, 0.13 mmol) and K₂CO₃ (37 mg, 0.26 mmol) in MeCN (5 mL) was heated at reflux with a CaCl₂ drying tube for 0.5 h, followed by addition of 2-(2-(2-azidoethoxy)ethoxy)ethyl 4-methylbenzenesulfonate (87 mg, 0.26 mmol). The resultant slurry was further heated at reflux for 24 h. The reaction mixture was allowed to cool to room temperature and the precipitate filtered off via suction filtration. The solvent was evaporated from the filtrate to dryness in vacuo and the crude solid was purified by purified via flash column chromatography (silica, 25% EtOAc: 75% n-hexane) to afford Compound 38 as a yellow solid (18 mg, 15%).

The name for Compound 40 is 11-(2-(2-(2-azidoethoxy)ethoxy)ethoxy)-8-(naphthalen-2-yl)-2,3,6,12-tetrakis(pentyloxy)triphenyleno[1,2-d]oxazole.

Compound 40 had the following characterisation data: ¹H NMR δ_{H}: (300 MHz, CDCl₃) 10.14 (1H, s), 8.82 (1H, s), 8.51 (1H, dd, J 8.6, 1.7), 8.06-8.03 (2H, m), 7.91-7.80 (5H, m), 7.57-7.55 (2H, m), 4.59 (2H, t, J 5.5 Hz), 4.41 (2H, t, J 6.7 Hz), 4.29-4.20 (6H, m), 4.15-4.10 (2H, m), 3.87-3.82 (2H, m), 3.69-3.57 (4H, m), 3.30-3.21 (2H, m), 2.05-1.93 (8H, m), 1.63-1.45 (16H, m), 1.05-0.98 (12H, m) ppm. ¹³C NMR δ_{c}: (100 MHz, CDCl₃) 161.7, 149.4, 148.8, 145.9, 145.4, 142.9, 140.8, 140.2, 134.7, 133.2, 129.1, 128.7, 128.0, 127.9, 127.6, 127.1, 126.9, 124.8, 124.7, 124.6, 124.0, 123.6, 123.4, 116.4, 112.7, 108.1, 107.2, 103.7, 103.6, 72.5, 70.7, 70.4, 70.1, 69.9, 69.8, 69.7, 69.0, 50.6, 29.32, 29.30, 29.17, 28.54, 28.46, 28.43, 22.75, 22.71, 14.27, 14.21 ppm. MALDI m/z: 912.9 ([M]⁺ 100%), 913.9 ([M+H]⁺ 90%). Elemental analysis Found: C, 73.36; H, 7.55; N, 6.12%. C₅₅H₆₈N₄O₈ requires C, 73.34; H, 7.51; N, 6.14%.

Method of Synthesisinq Compound 41

Compound 41 was synthesised using the following method. A slurry of Precursor 8 (100 mg, 0.13 mmol) and K₂CO₃ (37 mg, 0.26 mmol) in MeCN (5 mL) was heated at reflux with a CaCl₂ drying tube for 0.5 h followed by addition of 2-(2-(2-(2,5-dioxo-2,5-dihydro-1H-pyrrol-1-yl)ethoxy)ethoxy)ethyl 4-methylbenzenesulfonate (101 mg, 0.26 mg). The resultant slurry was further heated at reflux for 24 h. The reaction mixture was allowed to cool to room temperature and the precipitate filtered off via suction filtration. The solvent was evaporated from the filtrate to dryness in vacuo and the crude solid was purified by purified via flash column chromatography (silica, silica, 50% EtOAc: 50% n-hexane) to afford Compound 38 as a yellow solid (15 mg, 13%).

The name for Compound 41 is 2-(2-(2-((8-(naphthalen-2-yl)-2,3,6,12-tetrakis(pentyloxy)triphenyleno[1,2-d]oxazol-11-yl)oxy)ethoxy)ethoxy)ethan-1-amine.

Compound 41 had the following characterisation data: ¹H NMR δ_{H}: (300 MHz, CDCl₃) 10.13 (1H, s), 8.82 (1H, s), 8.50 (1H, dd, J 8.6, 1.7), 8.07-8.03 (2H, m), 7.91-7.81 (5H, m), 7.57-7.57 (2H, m), 4.58 (2H, t, J 5.5 Hz), 4.43 (2H, t, J 6.7 Hz), 4.28-4.20 (6H, m), 4.14-4.10 (2H, m), 3.86-3.82 (2H, m), 3.69-3.57 (4H, m), 3.17-3.19 (2H, m), 2.04-1.92 (8H, m), 1.63-1.44 (16H, m), 1.04-0.97 (12H, m) ppm. ¹³C NMR δ_{c}: (100 MHz, CDCl₃) 161.7, 149.4, 148.8, 145.9, 145.4, 142.9, 140.8, 140.2, 134.7, 133.2, 129.1, 128.7, 128.0, 127.9, 127.6, 127.1, 126.9, 124.8, 124.7, 124.6, 124.0, 123.6, 123.4, 116.4, 112.7, 108.1, 107.2, 103.7, 103.6, 72.3, 70.6, 70.3, 70.0, 69.9, 69.8, 69.7, 69.0, 42.5, 29.4, 29.3, 29.2, 28.5, 28.5, 28.4, 22.75, 22.7, 14.3, 14.2 ppm. MALDI m/z: 886.5 ([M]⁺ 100%), 887.6 ([M+H]⁺ 70%).

Method of Synthesisinq Compound 44

Compound 44 was synthesised using the following method. A solution of Precursor 2 (100 mg, 0.132 mmol), 3-fluorobenzoyl chloride (92 mg, 0.658 mmol) and N,N-diisopropylethylamine (0.1 mL, 0.574 mmol) in PhMe (5 mL) was heated to and held at reflux for 18 h under N₂. The reaction was cooled to room temperature and then evaporated to dryness in vacuo purified via flash column chromatography (silica, 60% CH₂Cl₂: 40% n-hexane) to afford an intermediate as a brown solid (19 mg, 18%).

The intermediate (3-fluoro-N-(2,3,6,7,10,11-hexakis(pentyloxy)triphenylen-1-yl)benzamide) (100 mg, 0.11 mmol) was dissolved in xylenes (10 mL) and Woollins reagent (117.8 mg, 0.22 mmol) was added to the flask. The reaction was stirred at reflux for 24 h before being cooled to room temperature causing the formation of a grey precipitate. The contents of the flask were filtered through filter paper and the filtrate recovered. The filtrate was evaporated to dryness and purified by column chromatography (silica, 40% dichloromethane: hexane) and (silica, 1% acetone: hexane) yielding 8-(3-fluorophenyl)-2,3,6,11,12-pentakis(pentyloxy)triphenyleno[1,2-d][1,3]selenazole as a yellow solid (1.8 mg).

The name for Compound 44 is 8-(3-fluorophenyl)-2,3,6,11,12-pentakis(pentyloxy)triphenyleno[1,2-d][1,3]selenazole.

Compound 44 had the following characterisation data: ¹H NMR δ_{H} (500 MHz, CDCl₃) 10.40 (1H, s), 8.00 (1H, s), 7.97 (1H, d, J 9.8 Hz), 7.95 (1H, s), 7.92 (1H, s), 7.90 (1H, d, J 7.8 Hz), 7.87 (1H, s), 7.49 (1H, dd, J 14.1, 8.5 Hz), 7.24-7.21 (1H, m), 4.45 (2H, t, J 6.8 Hz), 4.42 (2H, t, J 6.7 Hz), 4.28 (6H, dt, J 13.4, 6.6 Hz), 2.06-1.94 (10H, m), 1.61-1.44 (20H, m), 1.03-0.93 (15H, m) ppm. TOF LD⁺ m/z=860.4 ([M+2+H]⁺ 30%), 859.4 ([M+2]⁺ 60%), 858.4 ([M+H]⁺ 90%), 857.4 ([M]⁺ 100%), 856.4 ([M-2+H]⁺ 40%), 855.4 ([M-2]⁺ 60%), 854.4 ([M-3]⁺30%), 825.5 ([TpOxPhmF+MeOH]⁺), 793.4 ([TpOxPhmF]⁺).

Method of Synthesisinq Compound 45

Compound 45 was synthesised using the following method. Precursor 2 (190 mg, 0.25 mmol, 1 eq) and 4-methoxybenzoyl chloride (213 mg, 1.25 mmol, 5 eq) were dissolved in dry toluene (7 mL) and N,N-disopropylethylamine (0.2 mL, 1.25 mmol, 5 eq) was added. The solution was stirred and heated to reflux under a CaCl₂ drying tube for 2 h. The solution was then evaporated to dryness and the crude solid was heated to 240° C. for 10 mins. The crude product was recrystalised (dichloromethane: hexane, 1: 5) and then purified by silica plug (60% dichloromethane: hexane) to yield 8-(4-methoxyphenyl)-2,3,6,11,12-pentakis(pentyloxy)triphenyleno[1,2-d]oxazole as an off white solid (53 mg, 26%).

The name for Compound 45 is 8-(4-methoxyphenyl)-2,3,6,11,12-pentakis(pentyloxy)triphenyleno[1,2-d]oxazole.

Compound 45 had the following characterisation data: ¹H NMR δ_{H}: (300 MHz, CDCl₃) 10.15 (1H, s), 8.32 (2H, d, J 8.9 Hz), 7.93-7.87 (4H, m), 7.06 (2H, d, J 8.9 Hz), 4.46 (4H, t, J 7.0 Hz), 4.28-4.24 (6H, m), 3.93 (3H, s), 2.12-1.94 (10H, m), 1.65-1.43 (20H, m), 1.03-0.96 (15H, m) ppm. MALDI m/z: 804.9 ([M]⁺ 100%), 805.9 ([M+H]⁺ 70%), 806.9 ([M+1+H]⁺ 25%).

Method of Synthesisinq Compound 46

Compound 46 was synthesised using the following method. A solution of Precursor 1 (100 mg, 0.132 mmol), benzoyl chloride (92 mg, 0.658 mmol) and N,N-diisopropylethylamine (0.1 mL, 0.574 mmol) in PhMe (5 mL) was heated to and held at reflux for 18 h under N₂. The reaction was cooled to room temperature and then evaporated to dryness in vacuo purified via flash column chromatography 55 (silica, 60% CH₂Cl₂: 40% n-hexane) to afford an intermediate as a brown solid (19 mg, 18%).

The intermediate had the following characterisation data: ¹H NMR δ_{H}: (300 MHz, CDCl₃) 8.55 (1H, s), 8.45 (1H, s), 8.07 (2H, d, J 7.5 Hz), 7.78 (1H, s), 7.74 (1H, s), 7.72 (1H, s), 7.71 (1H, s), 7.59 (1H, d, J 7.0 Hz), 7.54 (2H, t, J 7.4 Hz), 4.28-4.12 (10H, m), 3.67-3.54 (2H, m), 2.00-1.85 (8H, m), 1.70-1.37 (20H, m), 1.34-1.06 (8H, m), 1.02-0.90 (12H, m), 0.83 (3H, t, J 7.0 Hz), 0.75 (3H, t, J 7.1 Hz) ppm. ¹³C NMR δ_{c}: (100 MHz, CDCl₃) 174.6, 151.0, 149.7, 148.7, 148.4, 148.4, 144.0, 135.1, 131.7, 130.9, 128.5, 127.9, 126.6, 124.7, 124.2, 123.0, 122.6, 122.0, 110.3, 108.1, 107.7, 106.8, 106.7, 73.4, 70.1, 70.0, 69.5, 69.3, 68.8, 32.1, 30.1, 29.9, 29.6, 29.4, 29.3, 28.7, 28.5, 28.5, 28.1, 22.9, 22.7, 22.6, 14.3, 14.3, 14.1 ppm. MALDI m/z: 863.3 ([M]+100%).

A solution of the intermediate (100 mg, 0.116 mmol) and Lawesson's Reagent (175 mg, 0.658 mmol) in PhMe (5 mL) was heated to and held at reflux for 48 h under N₂. The reaction was cooled to room temperature and then evaporated to dryness in vacuo. The solid was then heated and held at 240° C. for 15 mins under N₂. The crude black solid was then cooled to room temperature and purified via flash column chromatography (silica, 40% CH₂Cl₂: 60% n-hexane) to afford Compound 46 as a green solid (17 mg, 19%).

The name for Compound 46 is 2,3,6,11,12-pentakis(pentyloxy)-8-phenyltriphenyleno[1,2-d]thiazole.

Compound 46 had the following characterisation data: ¹H NMR δ_{H}: (300 MHz, CDCl₃) 10.51 (1H, s), 8.24-8.22 (2H, m), 7.92-7.89 (3H, m), 7.76 (1H, s), 7.53-7.52 (3H, m), 4.43-4.26 (10H, m), 2.10-1.95 (10H, m), 1.66-1.57 (10H, m), 1.53-1.47 (10H, m), 1.03-1.00 (15H, m) ppm. ¹³C NMR δ_{c}: (100 MHz, CDCl₃) 166.4, 152.5, 151.5, 150.2, 149.3, 148.3, 134.7, 130.9, 130.0, 129.3, 127.6, 125.7, 125.4, 124.7, 123.8, 119.0, 112.4, 108.9, 107.2, 106.9, 100.9, 70.3, 70.2, 69.7, 69.2, 69.1, 29.7, 29.6, 29.6, 29.5, 29.4, 28.8, 28.8, 28.8, 23.1, 23.0, 23.0, 23.0 23.0, 14.5, 14.5, 14.5, 14.5 ppm. MALDI m/z: 791.56 ([M]+100%).

Method of Synthesisinq Compound 47

Compound 47 was synthesised using the following method. A solution of Precursor 1 (100 mg, 0.132 mmol), 4-cyanobenzoyl chloride (109 mg, 0.658 mmol) and N,N-diisopropylethylamine (0.1 mL, 0.574 mmol) in PhMe (5 mL) was heated to and held at reflux for 18 h under N₂. The reaction was cooled to room temperature and then evaporated to dryness in vacuo. The crude brown solid was added to a solution of Lawesson's Reagent (175 mg, 0.658 mmol) in PhMe (5 mL) was heated to and held at reflux for 48 h under N₂. The reaction was cooled to room temperature and then evaporated to dryness in vacuo. The solid was then heated and held at 240° C. for 15 mins under N₂. The crude black solid was then cooled to room temperature and purified via flash column chromatography (silica, 40% CH₂Cl₂: 60% n-hexane) to afford Compound 47 as a yellow solid (5 mg, 5%).

The name for Compound 47 is 4-(2,3,6,11,12-pentakis(pentyloxy)triphenyleno[1,2-d]thiazol-8-yl)benzonitrile.

Compound 47 had the following characterisation data: ¹H NMR δ_{H}: (300 MHz, CDCl₃) 10.38 (1H, s), 8.31 (2H, d, J 8.4 Hz), 7.97-7.88 (4H, m), 7.79 (1H, d, J 8.5 Hz), 4.41-4.26 (10H, m), 2.06-1.95 (10H, m), 1.61-1.55 (10H, m), 1.51-1.44 (10H, m), 1.03-0.97 (15H, m) ppm. MALDI m/z: 816.9 ([M]+90%), 817.9 ([M+H]+ 100%).

Referring now to Table 1, there is shown luminescent data for Compounds 1 to 6.

TABLE 1
Luminescent data for Compounds 1 to 6

| c | ϵᵣ | Viscosity (cP) | | Compound 1 | Compound 2 | Compound 3 | Compound 4 | Compound 5 | Compound 6 |
|---|---|---|---|---|---|---|---|---|---|
| Ethyl acetate | 6.0 | 0.45 | Absorption λₘₐₓ (nm) | 281 | 270 | 272 | 275 | 272 | 253 |
| | | | Emission λₘₐₓ (nm) | 367 | 467 | 494 | 510 | 536 | 594 |
| | | | pSS (cm⁻¹) | 8300 | 15600 | 16500 | 16800 | 18100 | 22700 |
| | | | Φ | 0.18 ± 0.01 | 0.46 ± 0.04 | 0.55 ± 0.05 | 0.48 ± 0.04 | 0.51 ± 0.04 | † |
| | | | Brightness (M⁻¹ cm⁻¹) | 29 ± 5 | 51 ± 5 | 92 ± 9 | 56 ± 5 | 53 ± 5 | † |
| Octan-1-ol | 10.3 | 7.36 | Absorption λₘₐₓ (nm) | 281 | 271 | 272 | 275 | 270 | 255 |
| | | | Emission λₘₐₓ (nm) | 367 | 473 | 497 | 515 | 526 | 384 |
| | | | pSS (cm⁻¹) | 8300 | 15800 | 16600 | 17000 | 18000 | 13200 |
| | | | Φ | 0.30 ± 0.03 | 0.61 ± 0.06 | 0.71 ± 0.07 | 0.55 ± 0.05 | 0.56 ± 0.05 | † |
| | | | Brightness (M⁻¹ cm⁻¹) | 36 ± 7 | 64 ± 6 | 91 ± 9 | 67 ± 7 | 50 ± 5 | † |
| Acetonitrile | 37.5 | 0.38 | Absorption λₘₐₓ (nm) | 281 | 270 | 273 | 275 | 270 | 253 |
| | | | Emission λₘₐₓ (nm) | 367 | 492 | 524 | 543 | 592 | 630 |
| | | | pSS (cm⁻¹) | 8300 | 16700 | 17600 | 18000 | 20200 | 23650 |
| | | | Φ | 0.20 ± 0.02 | 0.46 ± 0.04 | 0.51 ± 0.05 | 0.36 ± 0.04 | 0.21 ± 0.02 | † |
| | | | Brightness (M⁻¹ cm⁻¹) | 12 ± 2 | 38 ± 4 | 44 ± 4 | 30 ± 3 | ‡ | † |

† No value could be obtained
‡ No value could be obtained due to poor solubility and therefore no ϵ data.

Referring now to Tables 2 to 4, there is provided luminescence data for pairs of compounds which are capable of absorbing at the same wavelength and emitting at different wavelengths.

TABLE 2
Luminescence data for pair 1

| | ε × 10³ (M⁻¹ cm⁻¹) at | | | Absorption | | | Emission | | | |
|---|---|---|---|---|---|---|---|---|---|---|
| | | 355 | 405 | λ_{Max} | % Abs at | % Abs at | λ_{Max} | pSS | | |
| Compound | λ_{Max} | nm | nm | (nm) | 355 nm^{a} | 405 nm^{a} | (nm) | (nm) | (cm⁻¹) | QY |
| Compound | 97 | 21 | 0 | 270 | 22 | 0 | 444 | 174 | 14000 | 0.40 |
| 45 | | | | | | | | | | |
| Compound | 103 | 13 | 14 | 272 | 12 | 14 | 558 | 286 | 18800 | 0.32 |
| 12 | | | | | | | | | | |

TABLE 3
Luminescence data for pair 2

| | ε × 10³ (M⁻¹ cm⁻¹) at | | | Absorption | | | Emission | | | |
|---|---|---|---|---|---|---|---|---|---|---|
| | | 355 | 405 | λ_{Max} | % Abs at | % Abs at | λ_{Max} | pSS | | |
| Compound | λ_{Max} | nm | nm | (nm) | 355 nm^{a} | 405 nm^{a} | (nm) | (nm) | (cm⁻¹) | QY |
| Compound | 97 | 21 | 0 | 270 | 22 | 0 | 444 | 174 | 14000 | 0.40 |
| 45 | | | | | | | | | | |
| Compound | 164 | 29 | 13 | 272 | 17 | 7 | 494 | 222 | 16500 | 0.55 |
| 3 | | | | | | | | | | |

TABLE 4
Luminescence data for pair 3

| | ε × 10³ (M⁻¹ cm⁻¹) at | | | Absorption | | | Emission | | | |
|---|---|---|---|---|---|---|---|---|---|---|
| | | 355 | 405 | λ_{Max} | % Abs at | % Abs at | λ_{Max} | pSS | | |
| Compound | λ_{Max} | nm | nm | (nm) | 355 nm^{a} | 405 nm^{a} | (nm) | (nm) | (cm⁻¹) | QY |
| Compound | 72 | 39 | 1.5 | 271 | 54 | 2 | 421 | 150 | 13150 | 0.70 |
| 22 | | | | | | | | | | |
| Compound | 164 | 29 | 13 | 272 | 17 | 7 | 494 | 222 | 16500 | 0.55 |
| 3 | | | | | | | | | | |

### EXAMPLE 1: VISUALISING A HUMAN LIVER

In Vitro Testing

To test the relative toxicity of tetrahydrofuran (THF) for liver cells, Huh-7 cells (hepatoma cell line used for a model of hepatocytes) were labelled with CMFDA (CellTracker Green, in DMSO, Invitrogen) and then treated with half log concentrations of THF (0.1%, 0.3%, 1%, 3%) for 1 hr. Cells were then imaged live using a Zeiss primovert fluorescence microscope.

The cell images indicated that at 0.1% and 0.3% THF there were no differences in the cell culture compared to the reference. At 1% and 3% THF an increasing number of dead cells were recorded.

Ex Vivo Testing

All fluorophores were testing using donated human tissue which had been previously rejected for transplantation. All reagents were diluted in pre-warmed Dulbecos Modified Eagles Medium (DMEM) or CO₂-independent media (both Invitrogen) to working concentrations and then perfused into pieces of liver for 45 min. Membrane and nuclear dyes were used simultaneously to visualise other cellular structures within the liver. Tissues were then imaged using multiphoton microscopy (825 nm). All fluorophores were used at 1 μg/ml (giving 0.1% of vehicle, being DMSO or THF). Testing of TpOx-2-Nap (green fluorophore) was done using DMSO as a solvent. Although this yielded limited solubility, the green fluorescence was still visible. A commercial red cell membrane dye (CMFDA) was used for comparison, having been perfused through a separate piece of tissue from the same donor. When using TpOx-Ph (blue fluorophore), THF was used as the solvent, which yield brighter and less punctate fluorescence.

FIG. 1 shows a multiphoton microscopy image (10) of an ex vivo liver perfused with TpOx-2-Nap. The green fluorophore was found to perfuse effectively throughout the human tissue and into the cells. Diffuse green fluorescence (12) was observed within the cytoplasm of the hepatocytes, bounded by the cell membranes (14) which were stained red by the commercial dye. Bright green spots (16) in the image indicated a significant uptake of the fluorophore in T cells. The tissue remained fluorescent over several hours, indicating that the fluorophore did not leak out of the cells.

FIGS. 2A and 2B show multiphoton microscopy images of an ex vivo liver perfused with TpOx-Ph (blue fluorophore). The bright areas indicate where the fluorophore has been taken up by the hepatocytes. In the centre of the images a black region (22) can be seen. This region (22) is the hepatic vein, which shows up as a black region in the image due to a complete lack of fluorophores inside the vein. The retention of the fluorophores within the hepatocytes provides good contrast between the vein and the hepatic tissue, enabling the vein to be observed over several hours.

These results demonstrate that the compounds described herein are suitable for live tissue imaging, since they permeate well into tissues, are capable of crossing the cell membrane, and provide bright emissions.

1-25. (canceled)

26. A composition for imaging a biological tissue or fluid comprising a compound of formula (A) and a biologically acceptable diluent or carrier, 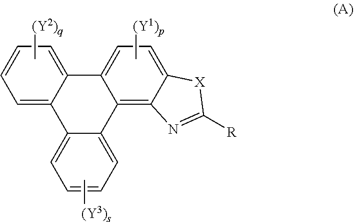
wherein X represents one of a nitrogen atom, an oxygen atom, a sulphur atom, a phosphorus atom, or a selenium atom;
R represents an aromatic group and/or an aliphatic group;
p is an integer of 1 to 2;
q and s are independently integers of 1, 2, 3, or 4;
Y¹, Y², and Y³ independently represent a hydrogen atom, a deuterium atom, a fluorine atom, a chlorine atom, a bromine atom, a substituted or unsubstituted alkyl group, a substituted or unsubstituted aryl group, a polyglycol group, an oxygen, a nitrogen atom, a cyano group, a nitro group; and/or
wherein two or more of Y¹, Y², and Y³ may combine together to form a condensed ring.

27. The composition of claim 26, wherein the luminescent compound is capable of crossing a cell membrane.

28. The composition of claim 26, wherein the luminescent compound is capable of being retained with cells for at least 10 minutes.

29. The composition of claim 26, wherein the luminescent compound preferentially accumulates in T cells.

30. The composition of claim 26, wherein the biologically acceptable diluent or carrier is selected from phosphate-buffered saline, saline, deionized water, blood, cell culture medium or plasma.

31. The composition of claim 26, wherein the luminescent compound is present in the composition at a concentration of from 0.1 to 20 μg/ml.

32. The composition of claim 26, wherein the composition comprises two or more different luminescent compounds, at least one of which has the general formula (A).

33. The composition of claim 26, wherein the composition comprises a first luminescent compound of formula (A) which emits light at a first wavelength and a second luminescent compound of formula (A) which emits light at a second wavelength, wherein the second wavelength which is longer than the first wavelength, wherein both the first and second compounds are capable of absorbing light at a third wavelength which is shorter than both the first and second wavelengths.

34. The composition of claim 26, wherein the luminescent compound of formula (A) is conjugated to a further molecule.

35. The composition of claim 26, wherein the composition comprises cells which have been dyed with the luminescent compound.

36. The use of a compound of formula (A), in a method of obtaining an image of a biological tissue or fluid using fluorescence microscopy.

37. The use of claim 36, wherein the biological tissue or fluid is in vivo, in vitro or ex vivo.

38. A method of obtaining an image of a biological tissue or fluid in vivo in a subject, the method comprising administering the composition of claim 26 to the subject and obtaining an image of the subject using fluorescence microscopy.

39. The method of claim 38, wherein the composition is administered topically, orally or parenterally.

40. A method of obtaining an image of a biological tissue or fluid previously obtained from a subject, the method comprising:
administering to, or contacting with, the biological tissue or fluid a luminescent compound of formula (A) 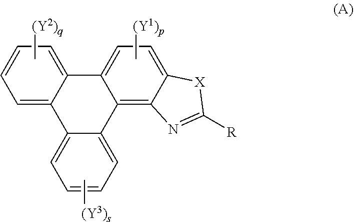
wherein X represents one of a nitrogen atom, an oxygen atom, a sulphur atom, a phosphorus atom, or a selenium atom;
R represents an aromatic group and/or an aliphatic group;
p is an integer of 1 to 2;
q and s are independently integers of 1, 2, 3, or 4;
Y¹, Y², and Y³ independently represent a hydrogen atom, a deuterium atom, a fluorine atom, a chlorine atom, a bromine atom, a substituted or unsubstituted alkyl group, a substituted or unsubstituted aryl group, a polyglycol group, an oxygen, a nitrogen atom, a cyano group, a nitro group; and/or
wherein two or more of Y¹, Y², and Y³ may combine together to form a condensed ring; and
obtaining an image of the biological tissue or fluid using fluorescence microscopy.

41. The method of claim 40, wherein the fluorescence microscopy is multiphoton fluorescence microscopy.

42. The method of claim 40, wherein the biological tissue is selected from blood, connective tissue, muscle tissue, nervous tissue, epithelial tissue, vasculature tissue, lymphoid tissue, tissues of the endocrine system, a gland or an organ.

43. The method of claim 38, for use in a diagnosis of a disease or condition in a subject, wherein the disease or condition is selected from: cancer, inflammation, oedema, cardiovascular disorders, ischemia, autoimmune disease, infection, skin disease, eye disease, neurological disorders or injury.

44. The method of claim 38, comprising subsequently determining the effectiveness of a treatment or therapy received by the subject.

45. The method of claim 40, comprising subsequently determining the effectiveness of a treatment or therapy received by the subject.
